(19) United States
(12) Patent Application Publication
(10) Pub. No.: US 2025/0261986 A1
(43) Pub. Date: Aug. 21, 2025

(54) ELECTROSURGICAL DEVICE AND METHOD OF USE

(71) Applicant: Meditrina, Inc., San Jose, CA (US)

(72) Inventors: Csaba TRUCKAI, Saratoga, CA (US); Zsolt TRUCKAI, Saratoga, CA (US); John H. SHADDUCK, Menlo Park, CA (US)

(73) Assignee: Meditrina, Inc., San Jose, CA (US)

(21) Appl. No.: 19/044,139

(22) Filed: Feb. 3, 2025

Related U.S. Application Data
(60) Provisional application No. 63/555,268, filed on Feb. 19, 2024.
(60) Provisional application No. 63/557,712, filed on Feb. 26, 2024.

(51) Int. Cl. A61B 18/14; A61B 18/00; A61B 18/12

(52) U.S. Cl. CPC A61B 18/14; A61B 2018/00208; A61B 2018/00601; A61B 2018/0091; A61B 2018/126; A61B 2018/1407; A61B 2018/1475; A61B 2218/007

(57) ABSTRACT

Bipolar electrosurgical devices and methods of use for cutting tissue, for example, in endoscopic urology and gynecology procedures. The electrosurgical device can comprise a handle and a small diameter, elongated shaft adapted for introduction through a working channel of an endoscope. The working end of the device has a form of loop electrode that is extendable outward from the shaft, a selected dimension for cutting tissue at a selected depth.

### CROSS-REFERENCE TO RELATED APPLICATIONS

This application is a non-provisional of U.S. Provisional Application Nos. 63/555,268, filed Feb. 19, 2024, and 63/557,712, filed Feb. 26, 2024, the entireties of which are incorporated by reference.

### FIELD OF THE INVENTION

The present invention relates to bipolar electrosurgical devices and methods of use for cutting tissue, for example, in endoscopic urology and gynecology procedures.

### SUMMARY OF THE INVENTION

Some aspects in accordance with principles of the present disclosure relate to a bipolar electrosurgical device. The electrosurgical device can comprise a handle and a small diameter, elongated shaft adapted for introduction through a working channel of an endoscope. The working end of the device has a form of loop electrode that is extendable outward from the shaft, a selected dimension for cutting tissue at a selected depth.

For example, a variation of an electrosurgical device include an electrosurgical resection device including: a handle coupled to an introducer member extending about a central axis to a working end, wherein the introducer member has an outer surface that defines an introducer envelope; an electrode shaft, an RF loop electrode carried by the electrode shaft and positioned at the working end extending in a transverse plane that is from 00 to 450 transverse to the central axis; a drive assembly, or deployment mechanism) within the handle and coupled to the electrode shaft, where the drive assembly is configured to be from the handle configured to move the electrode shaft to move the RF loop electrode between a first position within the introducer envelope for introduction into a working space and a second position radially outward from the introducer envelope for resecting tissue.

Variations of the device and method include a RF loop electrode that is carried at a distal end of an insulative shaft extending through a central passageway in the introducer member,

Additional variations of the device and method can include a deployment mechanism that is adapted to move the RF loop electrode between the first position and the second position by at least one of rotation of the insulative shaft and lateral movement of the insulative shaft.

Variations of the deployment mechanism can be further adapted to move the RF loop electrode axially between a distal position and a proximal position in the introducer member.

The deployment mechanism can be actuated by at least one of a motor drive and manual actuation.

Variations of the RF loop electrode can include an inflexible wire, or any similar structure.

In any variation, the RF loop electrode, when in the second position, can be positioned outward of the introducer envelope by at least 1 mm, 2 mm, 4 mm, or any distance therebetween. Unless specifically recited in the claims, the RF loop electrode can be positioned outward of the introducer envelope by any distance.

In some variations, the present disclosure includes an electrosurgical device further including an RF source coupled to the RF loop electrode, a negative pressure source communicating with the central passageway in the introducer member, a motor drive operatively coupled to the deployment mechanism, and a controller configured to control operating parameters of the RF source, negative pressure source and motor drive.

Variations of the devices can include including one or more accelerometers carried by the electrosurgical device on any portion of the device, and configured to send signals to the controller relating to movement of the electrosurgical device.

In some variations, the present disclosure includes an electrosurgical device wherein the introducer member includes an articulating section in the working end that is actuatable from the handle.

In some variations, the present disclosure includes an electrosurgical device wherein the RF loop electrode includes an active electrode and further including a return electrode carried in the working end.

In some variations, the present disclosure includes a method of resecting tissue in a working space in a patient, including: providing an introducer having an outer surface that defines an introducer envelope, the introducer extending about a central axis to a working end carrying an RF loop electrode that is moveable between a first position within the introducer envelope and a second position radially outward from a surface of the introducer that defines a selected cutting depth; introducing the working end in the first position through a working channel of an endoscope into the working space; moving the RF loop electrode to the second position; activating the RF loop electrode and translating the RF loop electrode through tissue at the selected cutting depth to thereby cut a tissue strip; and moving the RF loop electrode from the second position to the first position thereby mobilizing the tissue strip.

Variations of the devices in the present disclosure include a method wherein the RF loop electrode extends in a transverse plane that is from 0 degrees to 45 degrees transverse to a central axis of the introducer in the second position.

Variations of the methods include selecting the cutting depth by a mechanism in a handle portion of the introducer.

The methods and devices described herein can include an RF loop electrode that is moved between the first position and the second position in a rotational direction relative to the central axis of the introducer. Alternatively, or in combination, the RF loop electrode is moved between the first position and the second position in a transverse direction relative to the central axis of the introducer.

The RF loop electrode can be moved between the first position and the second position by at least one of a motor drive and manual actuation.

In some variations, the present disclosure includes a method wherein the RF loop electrode is translated through tissue by at least one of a motor drive and manual actuation.

In some variations, the present disclosure includes a method wherein a controller is configured to control movement of the RF loop electrode.

In some variations, the present disclosure includes a method of sending signals of movement of the introducer or RF loop electrode to the controller using an accelerometer, controlling movement of the RF loop electrode responsive to the signals using the controller.

### BRIEF DESCRIPTION OF THE FIGURES

FIG. 1 is a perspective view of an RF resecting device corresponding to the invention that has a handle connected to an elongate introducer assembly with a bi-polar loop electrode that can be rotatably extended and retraced from the introducer together with a block diagram of a negative pressure source, RF source, and controller operatively coupled to the device.

FIG. 2 is an enlarged perspective cut-away view of the working end of the RF device of FIG. 1.

FIG. 3A is a perspective view of the working end of the RF device of FIGS. 1 and 2 showing the RF loop electrode in a first retracted position relative to the introducer member of the device.

FIG. 3B is another view of the working end of FIG. 3A showing the RF loop electrode rotated to a position outward from the introducer to provide a first selected cutting depth.

FIG. 3C is another view of the working end of FIGS. 3A-3B showing the RF loop electrode rotated outward further to provide a second selected cutting depth.

FIG. 3D is a view of the working end of FIGS. 3A-3C showing the RF loop electrode rotated outward further to provide a third selected cutting depth.

FIG. 4 is a partly sectional side view of the working end of the device of FIGS. 1 to 3D showing the loop electrode rotatable in a transverse plane relative to the introducer axis.

FIG. 5 is a side view of another variation of a working end showing an RF loop electrode that is rotatable outward from an introducer, with the form of the electrode defining a non-transverse plane relative to the introducer axis.

FIG. 6 is a perspective view of another variation of a working end of an RF device wherein the RF loop electrode is rotatable outward from a slot in the introducer.

FIG. 7A is a schematic view of an initial step in the method of use for resecting a bladder tumor wherein an endoscope is introduced into a patient's bladder, followed by advancing the RF resecting device of FIG. 1 through a working channel of the endoscope, articulating the working end of the RF device and rotating the loop electrode outward from the introducer to provide a selected cutting depth.

FIG. 7B is an enlarged view of a subsequent step of the method of use wherein the RF loop electrode is energized, and the physician cuts a path into the targeted tissue with a margin around the tumor.

FIG. 7C is a view of another subsequent step of the method wherein the energized RF loop electrode cuts a path under the tumor as the physician moves the working end.

FIG. 7D is a view of the final step of the method wherein the energized RF loop electrode has cut a path fully under the tumor, and the physician rotates the energized loop electrode outwardly to cut, resect and mobilize the tissue chip.

FIG. 8 is a perspective view of another variation of an RF resecting device with a handle connected to an elongate introducer assembly wherein a bi-polar loop electrode is configured for lateral movement outward from the introducer using a cam mechanism together with a block diagram of a negative pressure source, RF source, electrical source, and controller operatively coupled to the device.

FIG. 9 is an exploded view of the working end of the RF device of FIG. 8, showing the RF loop electrode assembly and components of the cam mechanism for deploying the RF loop electrode.

FIG. 10A is a transparent perspective view of the working end of FIGS. 8 and 9 with the RF loop electrode in a retracted position.

FIG. 10B is a transparent view of the working end of FIG. 10A with the RF loop electrode in a laterally extended position.

FIG. 11A is a sectional view of the working end of FIGS. 8 and 9 with the RF loop electrode in a retracted position.

FIG. 11B is a sectional view of the working end of FIG. 11A with the RF loop electrode in a partially extended position outward from the introducer.

FIG. 11C is a sectional view of the working end of FIGS. 11A-11B with the RF loop electrode in a fully extended position outward from the introducer.

FIG. 12 is a perspective view of another variation of an RF resecting device with an elongate introducer carrying a bi-polar loop electrode together and an accelerometer in the handle coupled to a controller and motor drive for moving the loop electrode in response to signals from the accelerometer.

FIG. 13A is a schematic view of an initial step in an endoscopic method of using the resecting device of FIG. 12 in a fibroid resection procedure.

FIG. 13B is a subsequent step of the method of FIG. 13A showing advancement of the resecting device through a working channel of an endoscope and rotation outward of the loop electrode from the introducer member to a cutting position.

FIG. 13C is an enlarged view of a subsequent step of the method wherein the energized RF loop electrode cuts a path into tissue as the physician moves the working end.

FIG. 13D is the final step of the method wherein the energized RF loop electrode has cut a path under a strip of tissue, and the accelerometer signals the motor drive to rotate the loop electrode to fully resect and mobilize the tissue strip.

FIG. 14 is a perspective view of another variation of an RF resecting device with a deployable bi-polar RF loop electrode deployable from a window in the introducer member wherein a motor drive is configured to move the loop electrode outward, inward, and axially in the window.

FIG. 15A is a schematic view of an initial step in using the resecting device of FIG. 14, preparing to cut into tissue with a deployed, energized RF loop electrode.

FIG. 15B is a subsequent step of the method of FIG. 15A, wherein the energized RF loop electrode is motor-driven to cut an elongated path in tissue as the physician holds the working end in a stable position.

FIG. 15C is a subsequent step of the method of FIGS. 15A-15B, wherein the motor drive rotates the energized RF loop electrode to fully resect a strip of tissue and then return the de-energized loop electrode to a default position.

FIG. 16 is a perspective view of another variation of an RF resecting device with a working end carrying a deployable bi-polar loop electrode wherein the working end is motor-driven to axially reciprocate in an outer sleeve coupled to a handle.

FIG. 17 is a perspective view of another variation of an RF resecting device with a working end carrying a deployable bi-polar loop electrode wherein the introducer member comprises an extrusion of a polymeric material partly surrounded by a thin-wall metal sleeve.

FIG. 18 is an exploded view of the components of working end of the device of FIG. 17.

FIG. 19 is an enlarged perspective view of a working end similar to that of FIG. 17.

FIG. 20 is a perspective view of a variation of an RF loop electrode.

FIG. 21 is an end view of a working end similar to that of FIG. 19.

FIG. 22 is a cut-away view of the working end of FIG. 21.

FIG. 23A is a perspective view of another variation of a working end of an RF resecting device carrying an axially-extendable RF loop electrode.

FIG. 23B is a perspective view of the working end of FIG. 23A with the RF loop extended distally from an introducer sleeve illustrating the dimension of a tissue core than can be when the electrode is extended axially.

FIG. 24 is an end view of the working end of FIGS. 23A and 23B.

FIG. 25 is a perspective view of another variation of a working end of an RF resecting device with an RF loop electrode that is rotatable outward from the introducer sleeve and the introducer sleeve is axially-extendable.

FIG. 26A is a perspective view of another variation of a working end of an RF resecting device with an RF loop electrode that is rotatable outward from the introducer sleeve and a secondary fixed electrode with the introducer sleeve being axially-extendable and rotatable.

FIG. 26B is a view of the working end of FIG. 26A with the RF loop electrode rotated outward approximately 90° illustrating the dimension of a tissue core that can be cut when the introducer sleeve is extended axially.

FIG. 26C is another view of the working end of FIGS. 26A and 26B with the RF loop electrode rotated outward approximately 180° illustrating the dimension of a tissue core that can be cut when the introducer sleeve is extended axially.

FIG. 27 is a perspective view of another variation of a working end of an RF resecting device with a first arc-shaped electrode that is rotatable outward from the introducer sleeve and a secondary fixed electrode wherein the introducer sleeve is axially-extendable and rotatable to cut a core in tissue.

FIG. 28 is a perspective view of another variation of a working end of an RF resecting device with a first arc-shaped electrode that is rotatable outward from the introducer sleeve and a second arc-shaped fixed electrode wherein the introducer sleeve is axially-extendable and rotatable to cut a core in tissue.

FIG. 29 is a perspective view of another variation of a working end of an RF resecting device with a single arc-shaped electrode that is rotatable outward from the introducer sleeve wherein the electrode is rotatable by a motor drive and the introducer sleeve is axially-extendable and rotatable to cut a core in tissue.

FIG. 30 is a perspective view of another variation of a working end of an RF resecting device with the cutting sleeve de-mated from the outer introducer sleeve.

FIG. 31A is a perspective view of the working end of FIG. 30 with the RF loop electrode in a first position retracted into the bore of the outer introducer sleeve.

FIG. 31B is a view of the working end of FIG. 31A with the RF loop electrode in a second position with the cutting sleeve and/or RF loop electrode extended distally from the bore of the introducer sleeve.

FIG. 31C is a view of the working end of FIG. 31B with the RF loop electrode rotated outwardly to a cutting position outward of the introducer envelope.

FIG. 31D is a view of the working end of FIG. 31C illustrating a motor-driven spiral cutting path of the RF loop electrode to resect a cylindrical core in tissue.

FIG. 32 is a schematic view of a handle of the resecting device of FIGS. 31C-31D.

### DETAILED DESCRIPTION

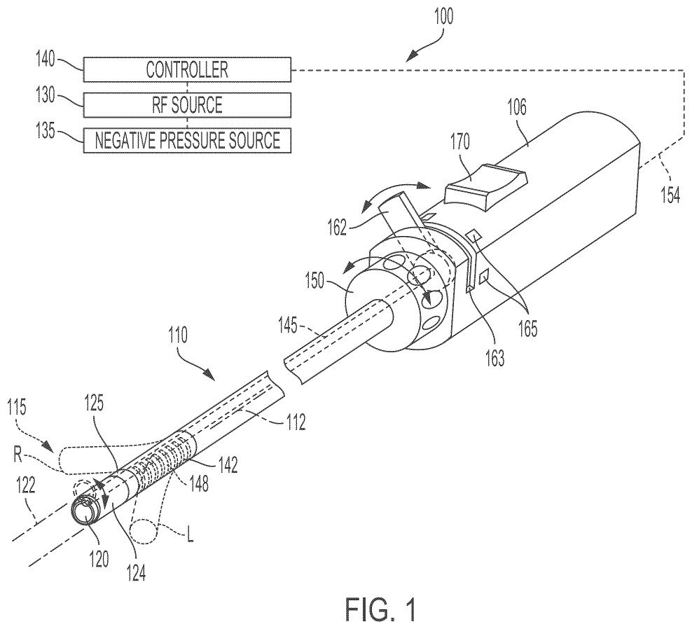

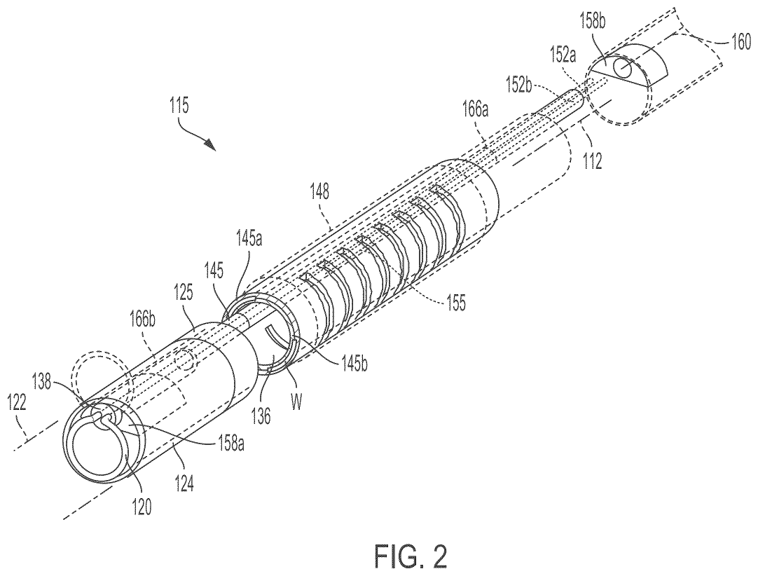

FIGS. 1 and 2 illustrate an electrosurgical tissue resecting device 100 comprising a handle 106 coupled to an elongated introducer member 110 extending about central axis 112 and having a diameter of 3 mm to 10 mm. The length of the introducer 110 can be highly elongated and suited for introduction through the working channel of an endoscope 114 (see FIG. 5A). In a variation, the introducer member 110 has a working end 115 shown in FIG. 2 carrying an RF active loop electrode 120 that is extendable outwardly by a deployment mechanism to be positioned outward of the cylindrical introducer envelope 122 of the introducer member 110. A distal portion of the working end 115 has an electrically insulative coating 124, with the adjacent proximal portion comprising a return electrode 125.

In the variation of FIG. 1, the RF resecting device 100 is operatively connected to a remote RF source 130 that is coupled to the active and return electrodes 120 and 125, a negative pressure source 135 that communicates with an interior passageway 136 in the introducer 110 (FIG. 2). The controller 140 is adapted to control the operating parameters of the RF source 130 and the negative pressure source 135.

The variation of FIG. 1 has an introducer member 110 with an articulating or deflecting section 142 in the working end 115, shown in more detail in FIG. 2. Typically, the deflectable section comprises an assembly of co-axial thin-wall slotted sleeves with outer sleeve 144a and inner sleeve 144b shown in the cut-away view of FIG. 2. Such slotted sleeve assemblies are known in the art, for example, as shown in U.S. Pat. No. 10,058,336 by Truckai et al., issued Aug. 28, 2018. The slotted sleeves 144a and 144b are fixed to one another with weld W (FIG. 2) at the distal end of the assembly, and the inner slotted sleeve 144b can be moved axially relative to the outer slotted sleeve 144a to deflect the deflectable section 142 as shown in dashed lines in FIG. 1 between left and right deflections indicated at L and R. A thin-wall flexible polymeric sleeve 148 encases the slotted sleeve assembly and is shown as a dashed line in FIG. 2. In this variation, a rotatable hub 150 at the proximal end of the introducer member 110 adjacent the handle 106 is rotated clockwise or counter-clockwise to deflect the working end 115 in the left and right deflections, L and R, as shown in FIG. 1. The rotatable hub 150 is operatively coupled to a proximal end to the inner sleeve 144b within the handle 106 with a helical coupling so that rotation of the hub 150 moves the inner sleeve axially relative to axis 112 in either the proximal or distal direction to deflect the working end as shown in FIG. 1.

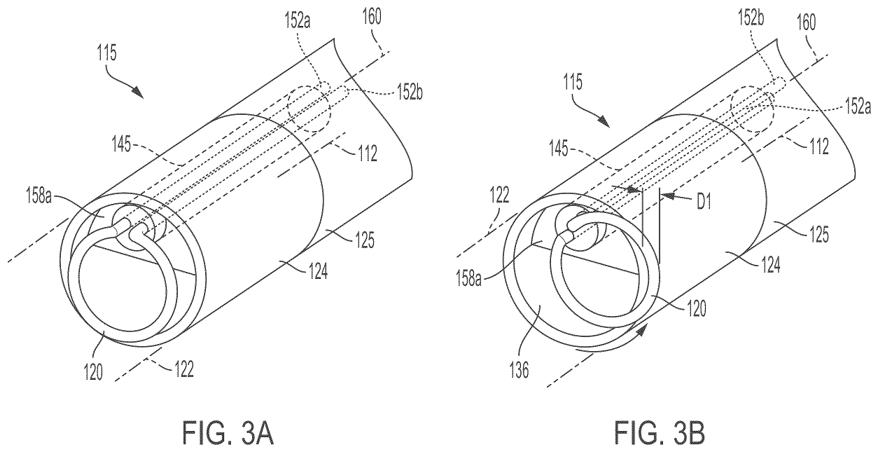

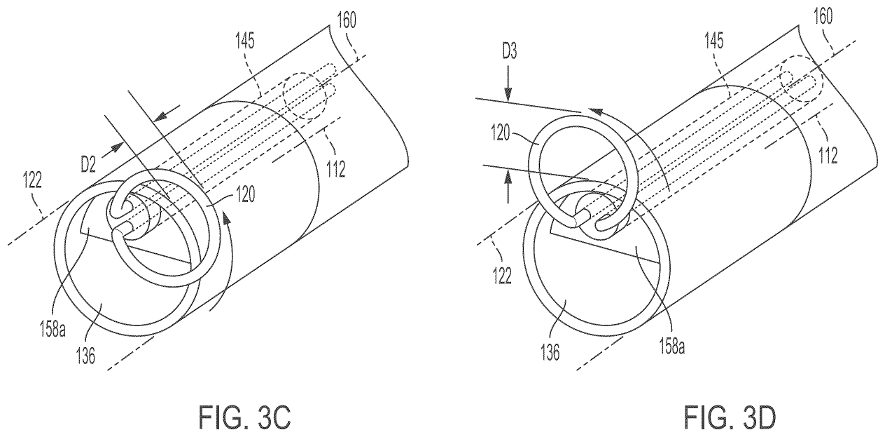

FIG. 2 further illustrates the components coupled to the moveable loop electrode 120 carried by the working end 115. The active loop electrode 120 extends distally from the distal end 138 of a rotatable, electrically insulated electrode shaft 145 that is carried within the interior passageway 136 of the introducer member 110. The electrode shaft 145 extends entirely through the passageway 136 to the handle 106 and is rotatable by a rotation mechanism that is further described below. The loop electrode 120 has first and second electrical leads 152a and 152b that extend through the electrode shaft 145 to the handle 106 and are coupled by an electrical cable 154 to the RF source 130 (FIG. 1). The electrode shaft 145 is configured with a flexible section 155 extending through the deflecting section 142 of the introducer 110 that comprises that a flexible drive shaft as known in the art. Such a flexible drive shaft can comprise a helical spring member, as is known in the art of flexible drive shafts. The electrical leads 152a, 152b within the electrode shaft 145 are small diameter insulated wires and are flexible. The electrode shaft 145 is rotatable within a plurality of collars fixed in the walls of introducer member 110 with collars 158a and 158b shown in FIG. 2. A plurality of such collars may be spaced apart in the passageway 136. The rigid portions of the introducer member 110 may have a continuous bore that carries the electrode shaft 145 in a wall of the inner sleeve 144b. As can be understood from FIGS. 1 and 2, the rotatable electrode shaft 145 has an off-center axis 160 that is offset from and parallel to the central axis 112 of the introducer member 110. Thus, it can be understood that rotation of the off-center electrode shaft 145 around axis 160 will rotate the loop electrode 120 from a first position within the cylindrical introducer envelope 122 of the introducer member 110 to selected other positions where the loop electrode 120 is positioned outward of the cylindrical introducer envelope 122 as shown in FIGS. 2 and 3B-3D. In FIG. 1, it can be seen that an actuator arm 162 is coupled to the electrode shaft 145 and is moveable in an arc in slot 163 in the handle to move the loop electrode 120 radially outwardly and inwardly. As can be seen in FIG. 1 the handle 106 can include markings 165 near the slot 163, indicating the position of the electrode 120. An actuator or power button 170 is provided in the handle 106 to energize the RF loop electrode 120 as described below in a method of use.

Referring again to FIG. 2, the distal portion of the outer sleeve 144a has the electrically insulative coating 124 that can comprise a polymeric or ceramic layer. In another variation, the distal portion of the outer sleeve 144a can be dipped in an epoxy to provide the insulative coating 124. The insulative coating 124 is adapted to space apart the RF loop electrode 120 from the return electrode 125, which can comprise exposed portions of the outer sleeve 144a. In FIG. 2, it also can be seen that the inner sleeve 144b may only extend within the outer sleeve 144a distally beyond the deflecting section 142 to the region of weld 144. Thus, the distalmost section of working end 115 would comprise only the outer sleeve 144a. Accordingly, the diameter of the electrode shaft 145 may have a smaller diameter portion 166a in the introducer section having both outer and inner sleeves 144a, 144b and a slightly large diameter portion 166b in the distalmost section of the working end 115 that comprises only the outer sleeve 144a.

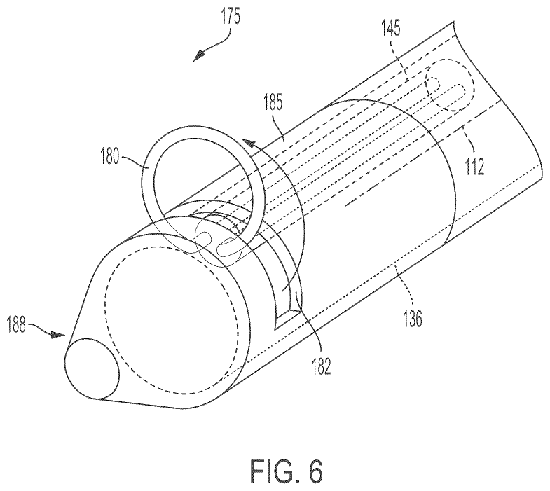

FIGS. 3A-3D are enlarged views of the working end 115 of the device 100 with the loop electrode 120 in various deployed positions with FIGS. 3B top 3D showing the electrode rotated outwardly from the introducer member 110 by the rotatable deployment mechanism. In FIG. 3A, the loop electrode 120 is in a first 0° position or introducer position where the electrode 120 does not extend outside the envelope 122 of the introducer and thus is configured for introduction through the working channel of an endoscope 114 (FIG. 6A). FIG. 3B shows the electrode shaft 145 after being rotated approximately 60° wherein the RF loop electrode 120 at its maximum outward point now extends outwardly of the introducer surface a dimension indicated as D1, which can be from 1 mm to 3 mm in this variation. Now, turning to FIG. 3C, the electrode shaft 145 is shown after being rotated by about 120° from the initial 0° position, where the depth of cutting depth of the RF loop electrode 120 is indicated at D2, which can be from 2 mm to 5 mm in this variation. FIG. 3D shows electrode shaft 145 after being rotated 180° from the initial 0° position, where the cutting depth of the RF loop electrode 120 is indicated at D3, which can be from 3 mm to 6 mm in this variation.

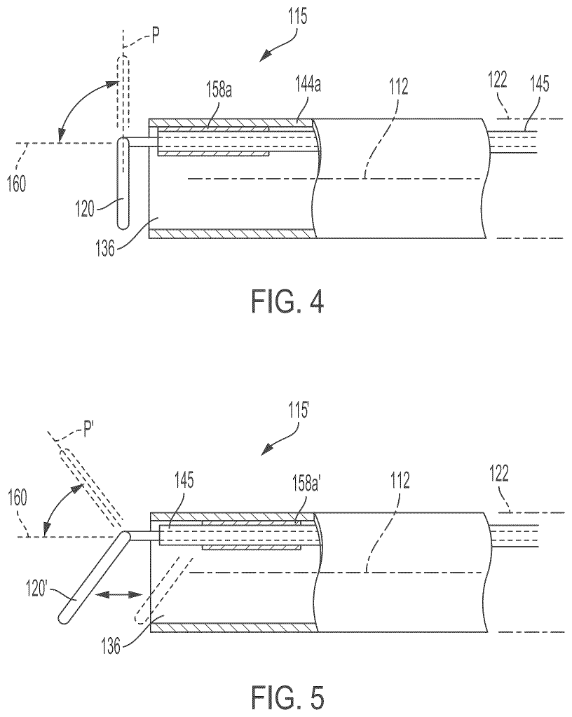

FIG. 4 is a side view of the working end 115 of FIGS. 2 and 3A-3D showing the RF loop electrode 120 angled from the electrode shaft 145 in a transverse direction or 90° direction from the axis 160 of the electrode shaft. When rotated as shown in FIGS. 3B-3D, the RF loop electrode 120 in all resecting positions rotates in a transverse plane P, as shown in FIG. 4.

FIG. 5 is a side view of a variation of a working end 115′ that has an RF loop electrode 120′ extending in plane P′ away from the electrode shaft 145 in a non-transverse angle in a range between 45° and 90° away from axis 160 of the electrode shaft 145. Thus, the scope of the invention and method of the invention includes an RF loop electrode that can be moved outward from the introducer envelope 122 and define a transverse plane P (FIG. 4) or a non-transverse plane P′ as in FIG. 5 that is less than 90° but more than 45° relative to axis 160 of the electrode shaft 145 or the parallel axis 112 of the introducer member 110. In FIG. 5, the loop electrode 120′ also is extendable and retractable in the interior passageway 136 in the first non-rotated position with collar 158a′ moved proximally in the passageway 136.

FIG. 6 illustrates another variation of a working end 175 that is similar to the working end 115 of FIGS. 1 to 4 described previously, except the variation of FIG. 6 is configured with a loop electrode 180 that rotates out of a slot 182 in an outer sleeve 185 that has a closed tip 188. In all other respects, the variation of FIG. 6 operates the same as the variation of FIGS. 1 to 4.

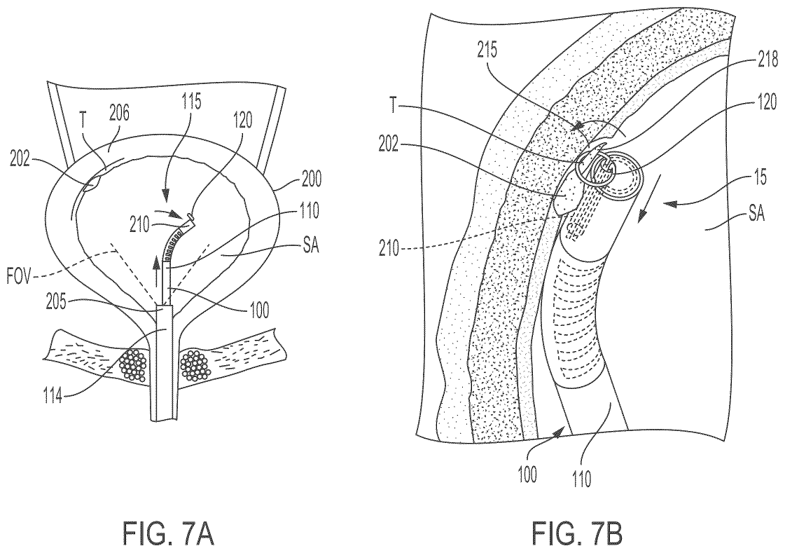

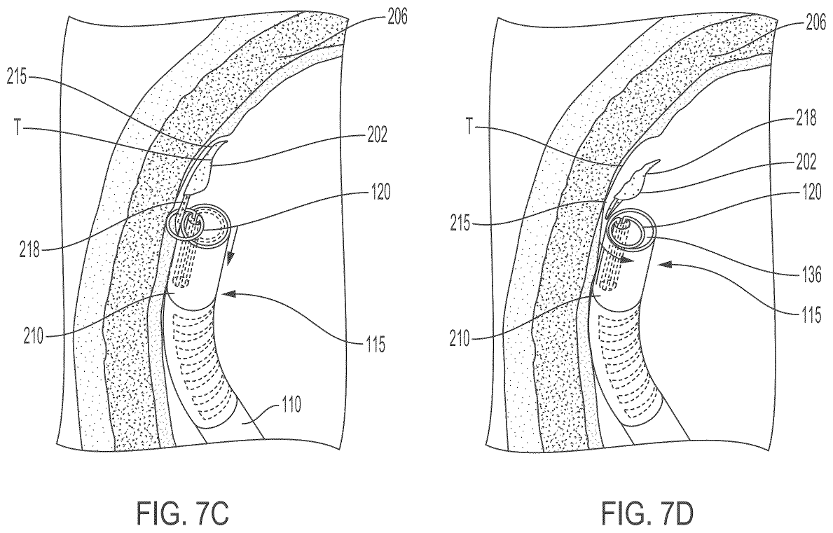

Now, turning to FIGS. 7A-7D, a method of the invention, is shown in an exemplary procedure of introducing the resecting device 100 of FIGS. 1 to 4 into a patient's bladder 200 to resect a bladder tumor 202. FIG. 7A shows an endoscope 114 being introduced into the patient's bladder 200 with saline SA introduced through the endoscope and within the bladder to distend the bladder as is known in the art. The introducer member 110 of the tissue resecting device 100 is introduced through the working channel of endoscope 114, wherein the field of view FOV of the image sensor 205 allows for viewing targeted tissue T of the bladder wall 206 and the bladder tumor 202. FIG. 7A further shows the working end 115 of the resection device 100 being articulated and the RF loop electrode 120 being moved into a cutting position outward of the outer surface 210 of the articulated introducer member 110, which can be any selected cutting depth as shown in FIGS. 3B to 3D that is determined by observations of the tumor 202 by the physician. The working end 115 is articulated to an angled position that is suited for alignment with the portion of the bladder wall 206 that has the tumor 202. The articulation of the working end 115 and the outward movement of the loop electrode 120 are accomplished by the physician moving the actuators in the handle 106 as described above (FIGS. 1 and 2).

As an example. FIG. 7B shows that the physician has selected a cutting depth as in FIGS. 3B to 3D, and the cutting depth is approximately the estimated depth of the tumor 202. In FIG. 7B, the physician then activates the RF loop electrode 120 by pressing power button 170 in the handle (FIG. 1) and cuts a path 215 into the bladder wall 206 near the tumor 202 until the surface 210 of the introducer member 110 is pressed against the bladder wall. In FIG. 7B, it can be understood that the physician translates the working end 115 in a proximal direction over the targeted tissue T, which includes a margin around the tumor 202 wherein the loop electrode 120 starts to cut a tissue chip 218 at the selected depth.

FIG. 7C illustrates a subsequent step wherein the physician translates the working end 115 further over the targeted tissue T with the loop electrode 120 cutting through or under the tumor 202 to further cut the tissue chip 218 and provide a margin on the opposing side of the tumor. Thus, in FIG. 7C, the physician has cut a path 215 completely under the bladder tumor 202. FIG. 7D then shows the physician rotating the RF loop electrode 120 from the outward cutting position toward the 0° position of FIG. 3A to thereby cut off the tissue chip 218 so that the tissue chip is entirely mobilized. The tissue chip 218 then may be extracted from the bladder 200 by suction through the interior passageway 136 in the introducer member 110 or by other means.

In FIGS. 7B to 7D, the resection of such a bladder tumor 202 is shown in a single cut with the RF loop electrode 120. It should be appreciated that in a typical procedure, the steps of the method shown in FIGS. 7B-7D can be repeated a number of times with suitable lesser cutting depths to resect a tumor with suitable margins.

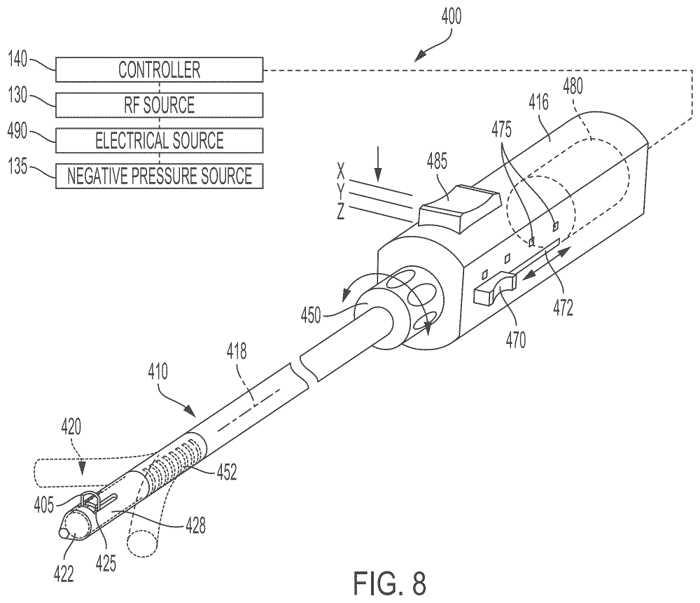

Now, turning to FIG. 8, another variation of a resecting device 400 is shown, which is similar to the device 100 of FIGS. 1 and 2, except the resecting device 400 has an RF loop electrode 405 that can be actuated to move outward from the introducer member 410 in a transverse direction by means of a cam mechanism described below. In FIG. 8, it can be seen that the resecting device 400 has a handle 416 coupled to the elongate introducer member 410 extending about longitudinal axis 418 to a working end 420. The working end 420 has a closed tip 422 as in the variation of FIG. 6 but could have an open end as in the variation of FIGS. 1 and 2. The RF active electrode 405 is movable outward from a partly circumferential slot 425 in the outer sleeve 428 of the working end 420. The resecting device 400 again is coupled to RF source 130, negative pressure source 135, and controller 140, as described above.

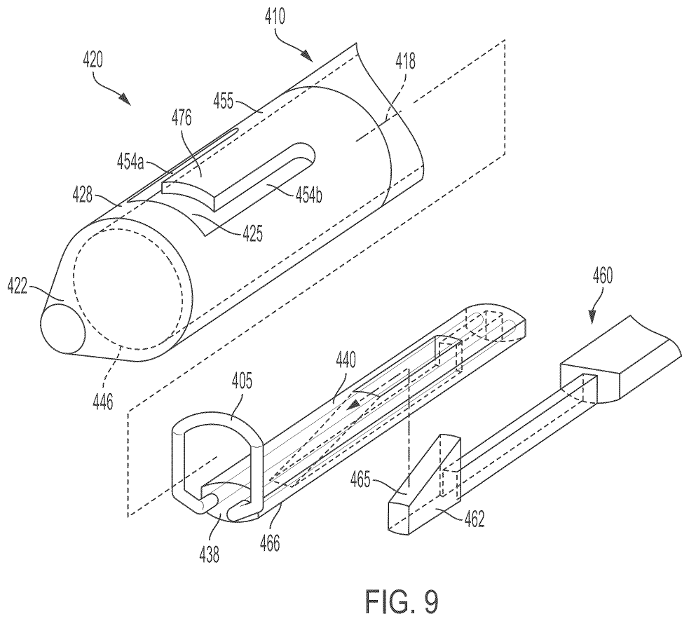

FIG. 9 is an exploded and partly transparent view of a distal portion of the introducer member 410, showing components of the cam mechanism that is adapted for moving the RF loop electrode 405 inward and outward from the introducer member 410. In FIG. 9, it can be seen that the RF loop electrode 405 is carried at the distal end 438 of an electrode shaft 440 that extends through a passageway 446 in the introducer member 410 to the handle 416 (FIG. 8). The introducer member 410 has a rotatable hub 450 that is adapted for articulating the articulating section 452 (FIG. 8) as described in the previous variation.

FIG. 9 shows that the outer sleeve 428 of the introducer member 410 is configured with the partly circumferential slot 425 from which the RF loop electrode 405 can be extended outwardly and retracted inwardly. The introducer member 410 further has axial slots 454a and 454b that are dimensioned to receive axial movement of the RF loop electrode 405 when the electrode has been moved to an outward cutting position. The distal section of the outer sleeve 428 has an insulative coating 455, as described in the previous variation.

In FIG. 9, it can be seen that a cam mechanism comprises an elongated shaft 460 that extends from the handle 416 to a distal wedge-shaped member 462 with an angled cam surface 465. The wedge-shaped member 462 is adapted to slide axially in a slot in the electrode shaft 440. It can be understood that when the wedge-shaped member 462 with its angled cam surface 465 is moved in the distal direction, the cam surface 465 will contact the adjacent surface 466 of electrode shaft 440 and move the loop electrode 405 transverse relative to the axis 418 of the introducer member 410. In FIG. 8, it can be seen that a slider grip 470 in the handle 416 is coupled to the elongated shaft 460 and is adapted to slide in slot 472 in the handle 416 to move the cam surface 465 distally to thereby move the RF loop electrode 405 outwardly from the introducer member 410. The handle 416 further includes indicators 475 at various positions of the slider grip 470, which correspond to different cutting depths of the RF loop electrode 405 as it extends outwardly from the surface 476 of the introducer member 410.

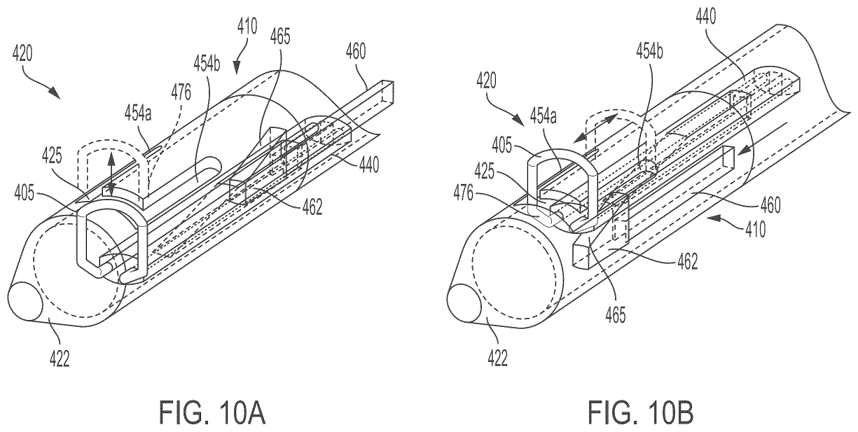

Now, turning to FIG. 10A, a transparent view of the working end 420 is shown with the elongate shaft 460 and cam surface 465 in a proximal position with the electrode shaft 440 and RF loop electrode 405 in a corresponding retracted position within the envelope of the introducer member 410. FIG. 10B shows the elongate shaft 460 and cam surface 465 in a distal extended position, which moves the electrode shaft 440 upwardly and the loop electrode 405 outwardly from the slot 425 in the introducer member 410. It can be understood that the loop electrode 405 can be extended outwardly from slot 425 in any selected dimension and maintained in such a position during use for any selected cutting depth.

Another aspect of the invention is shown in FIGS. 8 and 10B and comprises a motor drive 480 carried in handle 416 that is adapted to reciprocate the assembly of the electrode shaft 440 and the elongate shaft 460 carrying the cam surface 465. As can be seen in FIG. 10B, the loop electrode 405 can be reciprocated in axial slots 454a and 454b in the introducer member 410. Thus, a method of use allows the physician (i) to articulate the working end 420 as needed for a procedure, (ii) then energize the RF loop electrode 405 and press it into targeted tissue to cut a path, (iii) then maintain the working end 420 in a fixed location pressed against the targeted tissue, and (iv) then actuate the motor drive 480 to reciprocate the loop electrode 405 back and forth to cut an underlying path in the targeted tissue similar to that shown in FIGS. 6B and 6C. Thereafter, the physician can lift the loop electrode 405 away from the targeted tissue with the RF electrode energized to mobilize the tissue chip.

In the variation of FIGS. 8 to 10B, the device 400 can be actuated stepwise by first pressing the power button 485 (FIG. 8) from position X to Y, which energizes the RF loop electrode 405 and then can cut into the targeted tissue as described above. Next, the physician can press the power button 485 from position Y to position Z, which will reciprocate the energized loop electrode 405 in a single stroke back and forth. The physician can then release the power button 485 from position Z to position Y, and with the RF electrode 405 still energized, the physician can lift the working end 420 away from the tissue to cut the end of the tissue chip. In other variations, the controller 140 may reciprocate the loop electrode 405 back and forth a selected number of times to cut tissue. As can be seen in FIG. 8, the controller 140 also is coupled to an electrical source 490 that energizes the motor drive 480 in the handle 416. Thus, the controller 140 controls the RF source 130, the negative pressure source 135 as described previously, as well as the operating parameters of the motor drive 480. In some variations, the RF electrode can comprise a curved shape, an angled shape, a linear shape, or a combination thereof.

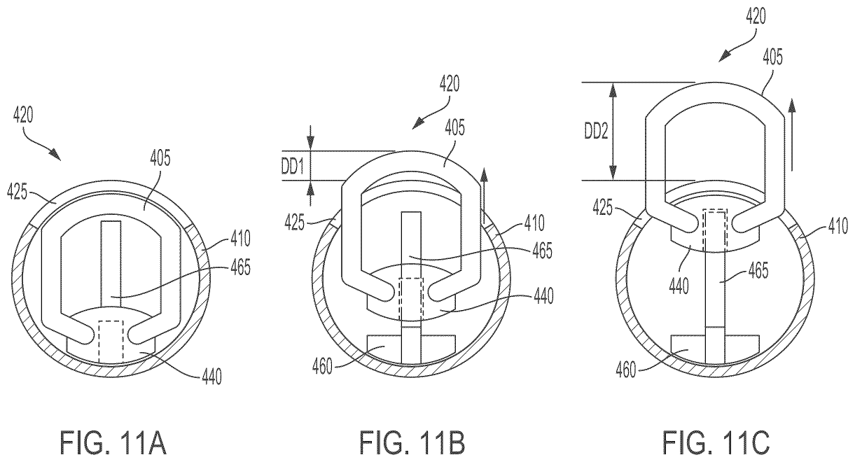

Now, turning to FIGS. 11A to 11C, sectional views of the working end of 420 of the device 400 of FIGS. 8 to 10B are shown with the RF loop electrode 405 in various positions. In FIG. 11A, the loop electrode 405 is in a constrained position within the envelope of the introducer member 410. The loop electrode 405 and electrode shaft 440 are urged into this initial constrained position by a leaf spring or other spring means (not shown) within the passageway 446 of the introducer member 410. FIG. 11B shows the cam surface 465 after being moved distally, which causes the electrode shaft 440 and loop electrode 405 to move outwardly to provide a selected cutting depth indicated by a DD1 that can range from 1 millimeter to 4 mm as described previously. FIG. 11C shows the cam surface 465 moving the electrode shaft 440 and loop electrode 405 outwardly for a maximum cutting depth DD2, which can range from 3 mm to 10 mm.

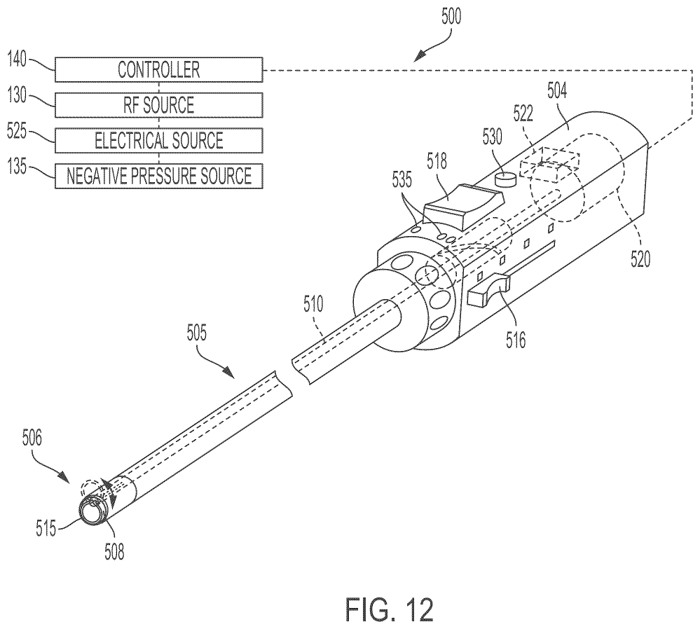

Now, turning to FIG. 12, another variation of a resecting device 500 is shown, which is similar to the device 100 of FIGS. 1 and 2, except it can be operated in first and second modes comprising a manual mode and a controller-assisted mode, respectively. The device 500 has a handle 504 coupled to an introducer member 505 extending to a working end 506. The introducer 505 has an interior passageway 508 carrying a rotatable electrode shaft 510, as described in previous variations. A distal RF loop electrode 515 is rotatable outward from the introducer member 505, as described in previous variations. In this variation, the manual mode of operation is the same as described above in the device of FIG. 1, with the drive assembly comprising a sliding grip 516 operable to move the loop electrode 515 outwardly to provide a selected cutting depth (see FIGS. 3B-3D). The mechanism of the sliding grip 516 includes electrical contacts (not shown), which are coupled to the controller 140 to signal the selected cutting depth to the controller for purposes described below relating to the second controller-assisted mode of operation. After selecting a cutting depth, the physician then would resect targeted tissue by (i) pressing the actuator button 518 to energize the loop electrode 515, (ii) then cutting a path into tissue and then translating the loop electrode through the targeted tissue to cut a strip of tissue, and (iii) then lifting the still-energized loop electrode 515 outwardly to thereby resect and mobilize the tissue strip (see FIGS. 7B-7D).

In the second controller-assisted mode, a motor drive 520 is used to rotate the loop electrode 515. As can be seen in FIG. 12, the handle 504 carries motor drive 520 that is coupled to the rotatable electrode shaft 510 and is configured to rotate the electrode shaft and loop electrode 515 in response to signals from an accelerometer 522. FIG. 12 shows accelerometer 522 carried in the handle 504, which is coupled to the controller 140. The controller 140 is also operatively coupled to an electrical source 525 that powers the electric motor drive 520. Referring to FIG. 12, the handle 504 has a switch 530 that allows the physician to toggle between the first and second modes of operation. The handle 504 further carries LEDs 535 that visually indicate selection of the first or second mode of operation.

In a variation, the accelerometer 522 sends signals to the controller 140 relating to movement of the device 500 or working end 506 during a resection procedure, wherein the controller 140 is configured with algorithms to determine any form of movement of the device 500, such as starting or stopping movement, or rate of change of movement of the device and the loop electrode 515. Responsive to such signals from the accelerometer 522, the controller 140 can change an operating parameter such as power delivered from RF source 130 to the loop electrode 515 and/or rotational movement of the loop electrode provided by the motor drive 520.

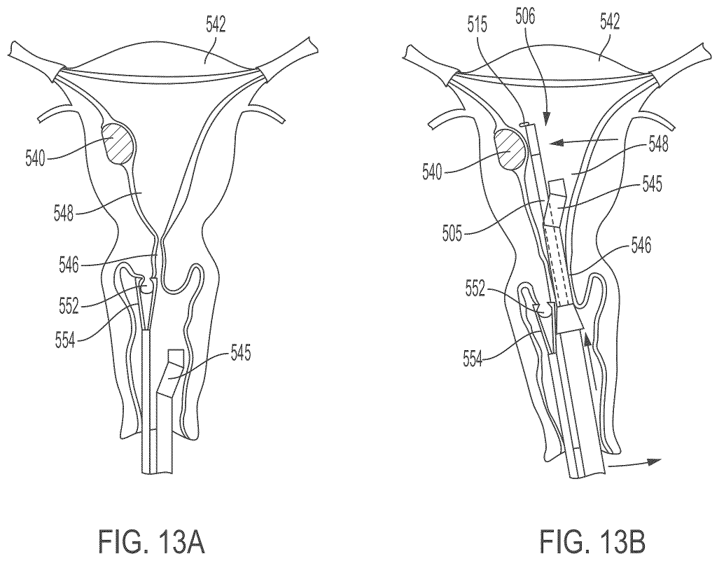

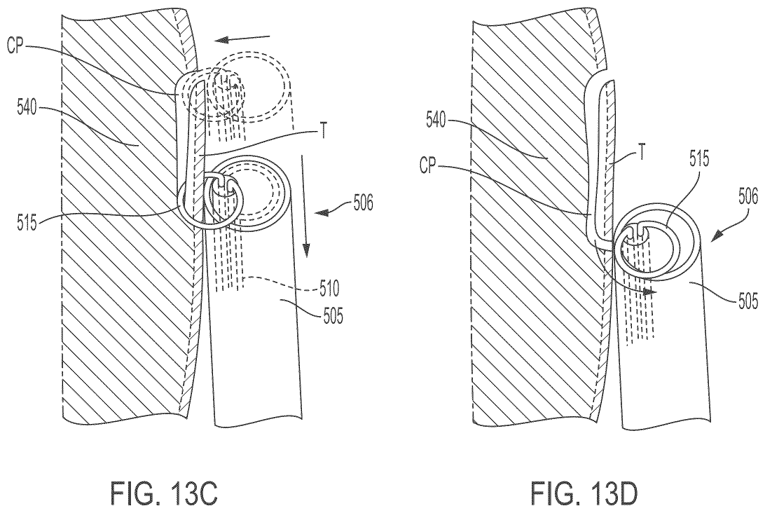

Now, turning to FIGS. 13A to 13D, the controller-assisted mode of operation, is illustrated in a gynecology procedure to resect a fibroid 540 in a patient's uterus 542. A variation of a method is shown using accelerometer 522 and controller 140 to assist in mobilizing a strip of tissue T (FIGS. 13C-13D). In the device of FIGS. 12 and 13A, the introducer member 505 does not include an articulating section as in previous variations. This is for convenience only, and the introducer member 505 optionally has such an articulating section. In FIGS. 13A to 13D, a resecting device 500 with a straight, rigid introducer member 505 is suitable for resecting a fibroid, and an articulating working end is typically not needed.

In FIG. 13A, the physician is preparing to introduce an endoscope 545 through the patient's cervical canal 546 into the uterine cavity 548, with the cervix 552 being stabilized with a tenaculum 554. The endoscope 545 is of the type having an expandable working channel as disclosed in commonly-owned U.S. Pat. No. 11,432,717 titled “Endoscope and Method of Use” authored by Truckai et al. FIG. 13B shows the endoscope 545 after introduction into the uterine cavity 548 with the resecting device 500 advanced through a working channel of the endoscope 545. FIG. 13B further illustrates the working end 506 of resecting device 500 being angled toward the fibroid 540, and the physician also adjusts the sliding grip 516 (FIG. 12) to select a cutting depth of the loop electrode 515. The physician has actuated the switch 530 in the handle 504 to operate in the second controller-assisted mode (FIG. 12).

Now, turning to the enlarged view of FIG. 13C, the physician energizes the RF loop electrode 515 by pressing power button 518 and cuts into the fibroid 540, or adjacent tissue, creating an inward cutting path CP and then translates the working end 506 proximally with the introducer surface in contact with tissue to extend the cutting path CP at the selected cutting depth through or under the fibroid 540 cutting a strip of tissue T. At the time the physician presses the power button 518, the controller logs signals from the accelerometer 522 which indicate movement of the working end 506 and loop electrode 515. A controller algorithm can determine that the working end 506 is being translated axially relative to the axis of the resecting device 500 and endoscope 545 (FIG. 13B). FIG. 13D then illustrates that the physician has cut an elongated cutting path CP at the selected depth and then stops movement of the working end 506. The control algorithm then in response to signals from the accelerometer 522 that movement of the electrode 515 has stopped, actuates the motor drive 520 to rotate the loop electrode 515 inwardly to cut off the end 550 of the tissue strip T. In this mode of operation, the loop electrode 515 remains energized as it moves to an inward position of FIG. 13D to cut off the tissue strip, and the controller algorithm then turns off the power to the electrode, and further the motor drive 520 rotates the de-energized loop electrode outwardly to the previously selected cutting depth. After these sequential steps, the working end 506 and loop electrode 515 are ready to repeat the cutting sequence described above to resect additional tissue strips to complete the resection of fibroid 540.

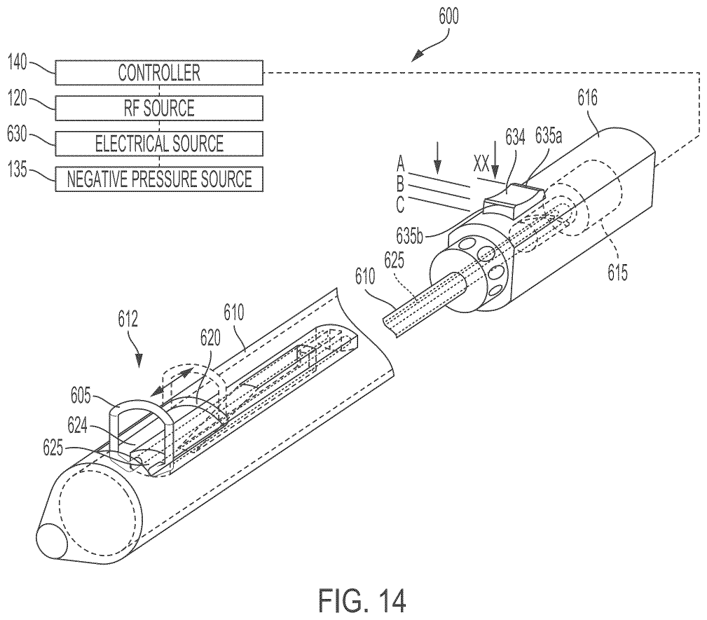

Now, turning to FIG. 14, another variation of a resecting device 600 is illustrated that is similar to device 400 of FIGS. 8 to 10B, except that the controller 140 is adapted to control an automated mechanism to reciprocate an RF loop electrode 605 and move the loop electrode 605 inwardly and optionally outwardly in the introducer member 610 of the device. This variation has an advantage in a method of use wherein the working end 612 is pressed against targeted tissue and held stationary while the loop electrode 605 is moved by a motor drive 615 to resect a strip of tissue in a partly automated manner. The method of using the previous variations typically required the physician to move or translate the loop electrode through tissue to resect a tissue strip.

More in particular, referring to FIG. 14, the resecting device 600 has a handle 616 coupled to introducer member 610 that extends to the working end 612 with an elongated window 620 therein. The loop electrode 605 reciprocates and moves outwardly and inwardly from window 620. The introducer 610 has an interior passageway 624 carrying an electrode shaft 625 of the same type as in device 400 of FIGS. 8 and 9. The electrode shaft 625 and loop electrode 605 of FIG. 14 also is actuated by the same cam mechanism as fully described above and shown in FIGS. 8 and 9. The handle 616 carries the motor drive 615 powered by electrical source 630 that is configured to move the assembly of the electrode shaft 625, cam mechanism, and loop electrode 605 to resect strips of tissue. The motor drive 615 has an output shaft coupled to linear drive and rotation mechanisms known in the art to move the electrode shaft assembly together with the cam mechanism to axially translate the entire loop electrode assembly.

In a variation, an actuator button 634 in the handle 616 is configured to perform multiple functions, including selecting the cutting depth of the loop electrode 605. In FIG. 14, the proximal end 635a of the actuator button 634 can be depressed multiple times to toggle through a plurality of cutting depths that are adjusted by the motor drive 615, for example, from two to five different selected cutting depths of 1 mm to 5 mm. A plurality of LEDs can be provided (not shown) on the handle 616 to indicate the selected cutting depth, while the physician can also observe the cutting depth of the loop electrode endoscopically. In a non-deployed or default position, the loop electrode 605 and electrode shaft 625 are positioned distally and inward relative to the window 620.

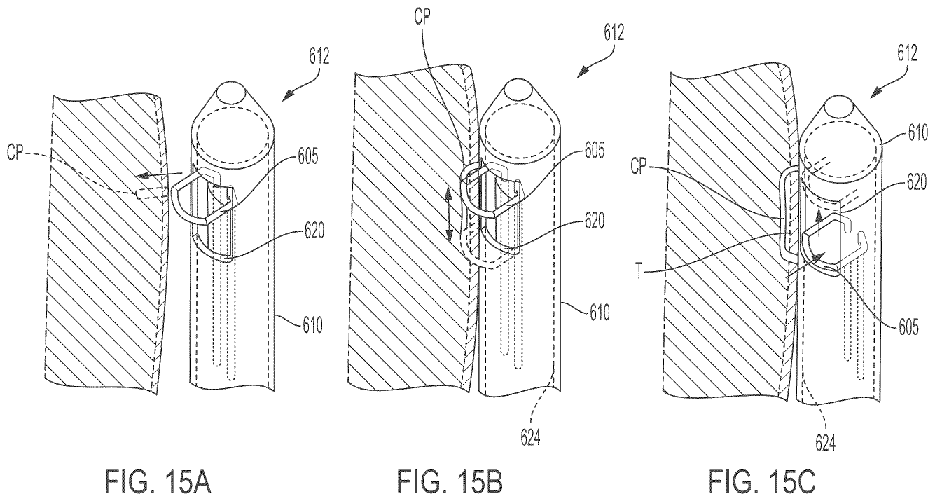

Turning to FIGS. 15A to 15C, a method of use is illustrated wherein the working end 612 is positioned in a saline-distended working space WS. In FIG. 15A, the physician uses the actuator button 634, as described above, to select a cutting depth. Thereafter, the physician depresses the distal end 635b of the actuator button 634 from A to B, which moves the loop electrode 605 outwardly to the selected cutting depth and energizes the loop electrode (FIG. 14). The physician then moves the loop electrode 605 into tissue to cut a cutting path CP as indicated by the dashed line in FIG. 15A until the outer surface of the introducer contacts tissue, as depicted in FIG. 15B.

FIG. 15B illustrates a subsequent step of the method where the physician depresses the distal end 635b of the actuator button 634 from B to C, which signals to controller 140 to actuate the motor drive 615 to move the loop electrode 605 proximally to extend the cutting path CP at the selected cutting depth. FIG. 15C illustrates a continued cutting step wherein the motor drive 615 moves the loop electrode 605 inwardly into the window 620 of introducer member 610 to fully resect and mobilize the tissue strip T as the loop electrode 605 reaches its retracted, proximal position. Typically, the strip of tissue T would be aspirated into the internal passageway 624 by the negative pressure source 135 and extracted to a tissue trap (not shown) as is known in that art. FIG. 15C further illustrates that the controller 140 would (i) de-energize the loop electrode 605 when the electrode reaches its retracted, proximal position and then (i) operate the motor drive 615 to move the loop electrode 605 to its distal default position. Thereafter, the physician repeats the above steps to resect additional tissue strips to complete a procedure.

The resecting device 600 of FIG. 14 also can be used with a controller 140 configured to operate the loop electrode 605 in a more fully automated manner. For example, the system could be configured so that the physician positions the working end 612 and window 620 against the targeted tissue and then press the actuator button 634 to initiate the following steps: (i) the controller 140 energizes the loop electrode 605, (ii) the motor drive is then activated so the cam mechanism moves the loop electrode 605 outward from the window 615 to cut a cutting path transversely into the tissue to the selected cutting depth, (iii) the motor drive 615 is then activated to cause axial translation of the loop electrode assembly to extend the cutting path axially within the tissue, (iv) the motor drive then retracts the loop electrode 605 inward into the window 615 to cut off the strip of tissue; and (v) the electrode is de-energized, and motor drive returns the loop electrode to the default, distal position. Thereafter, the above steps can be repeated in the same sequence. In this method, pressing the actuator button 634 could initiate a single sequence of steps as described above or simply continue the series of steps until the power button is released. This method would allow that physician to resect multiple strips of tissue without having to translate the resecting device inward and outward of the working channel in the endoscope.

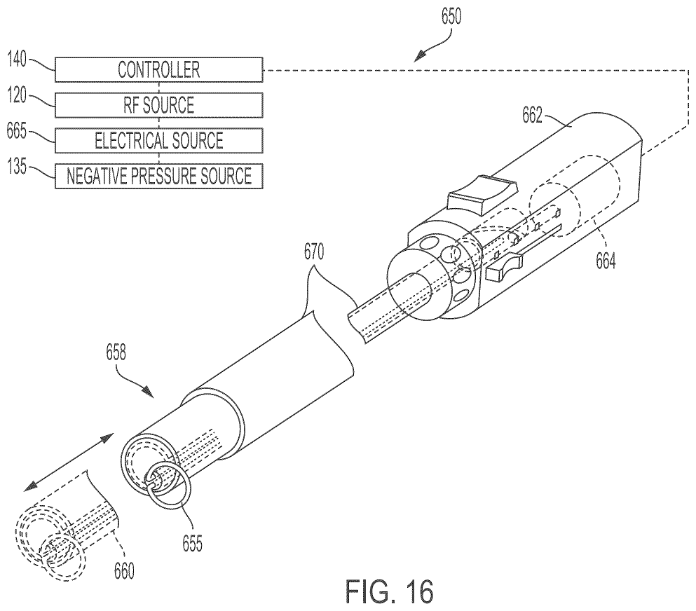

FIG. 16 illustrates another variation of a resecting device 650, which is similar to the device 100 of FIGS. 1 to 3D with an RF loop electrode 655 that can be rotated outwardly and inwardly from the working end 658 relative to the envelope of the introducer assembly 660. The variation or FIG. 16 includes a handle 662 carrying a motor drive 664 that is powered by electrical source 665. In this variation, the motor drive 664 is adapted to reciprocate the entire working end 658 in an outer sleeve 670 of the introducer assembly 660. This variation has an advantage in a method similar to that of device 600 of FIGS. 14-15, wherein the physician can move the loop electrode 655 into and out of a tissue surface without manually moving the working end 658 axially. Instead, the variation of FIG. 16 uses the motor drive 664 to reciprocate the working end 658 and loop electrode 655. In this method of use, the physician can maintain the device axially motionless relative to the endoscope (cf. FIGS. 13C-13D), which is an advantage. In this variation, the selected cutting depth is adjusted by the sliding grip 672, as described previously. In a variation, the reciprocation mechanism in the handle 662 can be a form of linear actuator known in the art with a reversible motor drive.

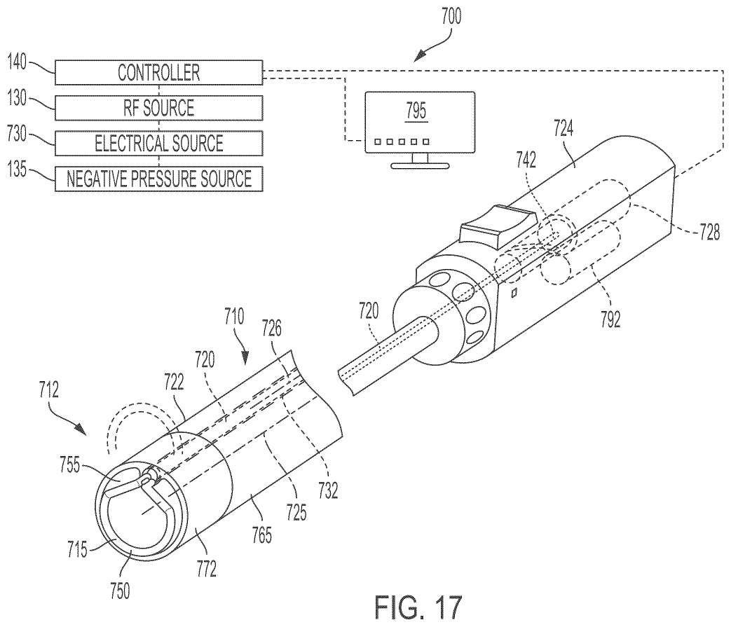

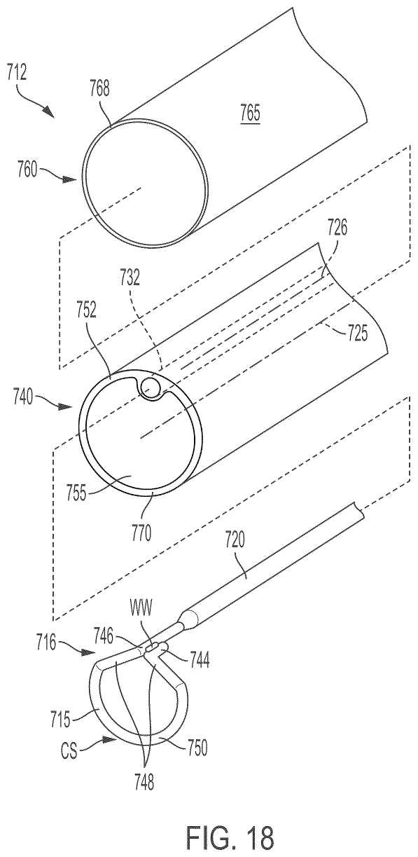

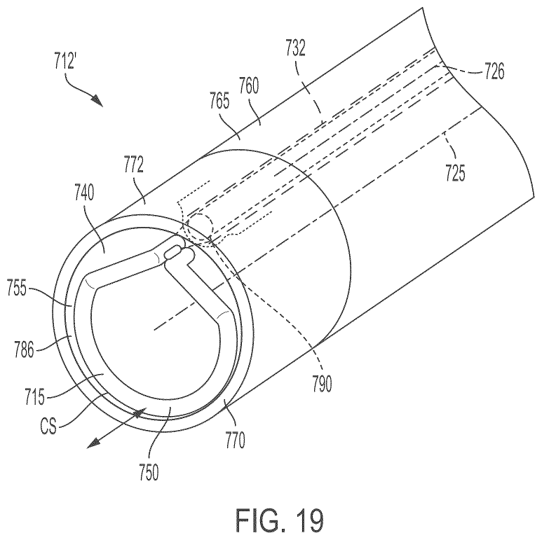

Now, turning to FIGS. 17 and 18, another variation of a resecting device 700 is illustrated that is similar to device 100 of FIGS. 1 to 3D, except that the introducer member 710, working end 712, and RF loop electrode 715 are designed to maximize the cutting depth of the loop electrode 715 when deployed relative to the diameter of the introducer member. As can be seen in FIG. 18, a unitary electrode member 716 comprises loop electrode 715 at the distal end of an electrode shaft 720 that extends through the introducer member 710. The increased cutting depth of the loop electrode 715 is attained by reducing the diameter of the electrode shaft 720 and positioning the electrode shaft 720 close to the introducer envelope 722, which is defined by the outer surface of the introducer member 710. The method of using the resecting device 700 is similar to the methods described above.

More, in particular, FIG. 17 illustrates the resecting device 700 with a handle 724 coupled to introducer member 710 that extends about longitudinal axis 725 to the working end 712. The loop electrode 715 is shown in FIG. 17 in a default or inward position for introduction into a working space and is adapted for deployment outwardly from the introducer member 710 and introducer envelope 722 by rotation of the electrode shaft 720 about its axis 726 as described previously. The deployment mechanism comprises a motor drive 728 carried within the handle 724 configured to rotate the electrode shaft 720, but manual deployment is also possible, as in previous variations.

The motor drive 728 in the handle is powered by electrical source 730 that is similar to previous variations. The resecting device 700 and motor drive 728 are again operatively connected to an RF source 130, negative pressure source 135, and controller 140 that function as described in previous variations.

Now referring to FIG. 18, the components of the introducer member 710 and working end 712 are shown in exploded view. In this variation, the electrode member 716 is configured with loop electrode 715 that extends distally from electrode shaft 720. FIGS. 17 and 18 show the electrode shaft 720 extending through a channel 732 in an extruded central core sleeve 740 of the introducer member 710. The electrode shaft 720 extends through channel 732 to the handle 724, where a proximal end 742 of the electrode shaft is operatively coupled to the motor drive 728 (FIG. 17). In a variation shown in FIG. 18, the electrode member 716 can comprise a tungsten wire with shaft 720 having a first diameter and the distal loop electrode 715 having a second reduced diameter. In a variation, the introducer member 710 is elongated for introduction through a working channel of an endoscope, and it can be understood that the electrode shaft 720 is required to resist twisting and, therefore, has a suitable torque-resistant diameter which is greater than 0.015″ and often greater than 0.018″. In this variation, the tungsten wire portion comprising the loop electrode 715 has a diameter less than 0.015″ and often less than 0.012″. The unitary electrode member 716, as shown in FIG. 18 can be fabricated by providing a suitable tungsten wire or other suitable metal wire having the diameter of the electrode shaft 720 described above and then grinding a distal portion of the wire to a dimension described above for a loop electrode 715 followed by forming the circular shape of the loop electrode 715.

In the variation of FIG. 18, the reduced diameter of the loop electrode 715 reduces the active electrode surface area and thus reduces the power requirements from RF source 130 to ignite a plasma for cutting tissue. In the variation shown in FIG. 18, the loop electrode 715 extends in 360°. and a weld WW fuses the tail end 744 of the loop with initial section 746 of the loop. It can be seen that in FIG. 18 that the loop electrode 715 has a cylindrical shape CS at least in part and leg portions 748 that extend to axial the portion of the electrode shaft 720. The circular shape CS extends from 180° to 360° around the loop shape and comprises the active electrode 750. The leg portions 748 of electrode 715 may be covered with an insulative material such as an epoxy or heat shrink material (not shown) to further reduce the active electrode surface area. In a typical variation, the bare wire portion of loop electrode 715 comprising the active electrode 750 extends at least 180° around the circular shape CS of the loop.

Referring to FIG. 18, the central core sleeve 740 of the introducer member 710 comprises an extruded polymeric material such as FEP. The diameter of channel 732 in the wall 752 of the core sleeve 740 is dimensioned to allow rotation and optional axial movement of the electrode shaft 720 therein. The central passageway 755 in the core sleeve 740 communicates with the negative pressure source 135. In FIGS. 18 and 19, it can be understood that the combination of small diameter electrode shaft 720 and extruded core sleeve 740 allows for an increased cutting depth when the loop electrode 715 is rotated 180° out of its default inward position of FIG. 17.

Referring again to FIG. 18, the extruded core sleeve 740 is disposed within a thin metal outer sleeve 760, such as stainless steel, that provides stiffness for the assembly comprising the elongated introducer member 710. In this variation, the introducer member 710 is configured as a rigid assembly and is not adapted for articulation as in some previous variations. The metal outer sleeve 760 can comprise a stainless steel tube with a wall thickness of less than 0.006″ or less than 0.005″ to provide a suitable stiffness to the introducer in a variation where the introducer has an outer diameter of less than 6 mm or less than 5 mm. In the variation of FIGS. 17 to 19, the outer sleeve 760 comprises a return electrode 765. In an assembled variation shown in FIG. 19, the outer sleeve 760 has a distal end 768 that is spaced apart from the distal end 770 of the core sleeve 740 to provide an insulative material between the loop electrode 715 and the return electrode 765 during use. As can be seen in FIGS. 17, 19, and 22, an insulative circumferential band 772 is provided around the distal end 770 of the core sleeve 740, which can comprise a heat shrink material, epoxy, or the like. The axial length of the insulative circumferential band 772 is at least 1.0 mm or at least 2.0 mm and often greater than 4.0 mm.

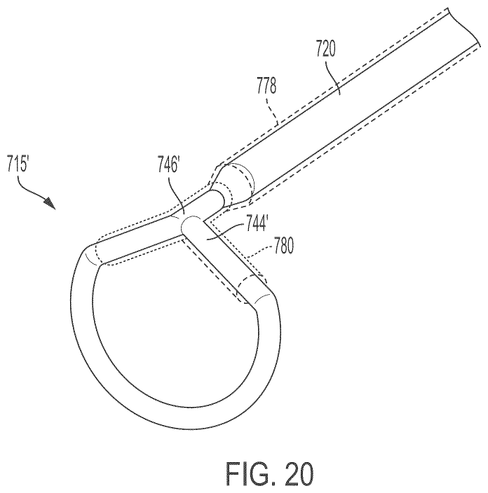

FIG. 20 illustrates a variation of loop electrode 715′ wherein the tail end 744′ of the loop is not welded to the initial loop section 746′ as in the variation of FIG. 18. In FIG. 20, the tail end 744′ and initial section 746′ are bonded together with an epoxy material 780 molded around a portion of electrode 715′ to provide an insulative layer and to connect the tail end 744′ and initial section 746′ of the loop electrode. In this variation, the electrode shaft 720 is also shown encased in a heat shrink member 778 to add to the insulative properties of the electrode shaft 720.

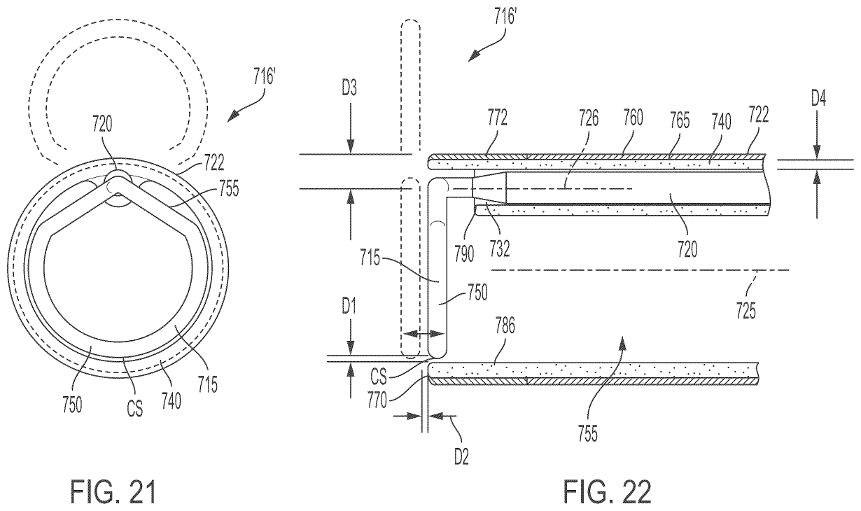

Now, turning to FIGS. 19, 21, and 22, another aspect of the working end 712′ of a resecting device is shown. In this variation, the working end 712′ includes a loop electrode 715 configured for axial extension and retraction in the central passageway 755 of the working end, wherein the outer surface 785 of the circular shape CS of loop electrode 715 is closely matched in dimension to the surfaces 786 of passageway 755 so that when the loop electrode 715 is retracted into the passageway 755, any eschar sticking to the loop electrode 715 will be scraped away by surface 786. FIG. 22 shows the radial distance D1 between the loop electrode 715 and surface 786 is minimal and is often less than 0.5 mm. Similarly, as can be seen in FIGS. 21 and 22, when the loop electrode 715 is extended outwardly from the passageway 755, rotation of the loop electrode from any deployed position to the default, inward position will scrape eschar from the proximal surface 788 of the loop electrode 715. FIG. 22 shows the axial distance D2 between the loop electrode 715 and distal end 770 of the core sleeve 740 is minimal and is often less than 0.5 mm. As can be seen in FIGS. 19 and 22, the distal end 790 of channel 732 carrying the electrode shaft 720 is recessed in the core sleeve 740 to allow retraction of the loop electrode 715 into the passageway 755.

Referring back to FIG. 17, it can be seen that the handle 724 carries a second motor drive 792 that is adapted for causing axial movement of the loop electrode 715 as shown in FIGS. 19 and 22. Thus, in a variation, the handle 724 carries the first motor drive 728 for rotational deployment of the loop electrode 715, and the second motor drive 792 is configured for the axial extension and retraction of loop electrode 715 from the passageway 755. In other variations, a single motor can have mechanisms adapted for performing both rotational and axial movements.

In a variation, the motor drives 728 and 792 comprise encoder motors, which send signals to the controller 140 as to the degree of rotation of the motor shaft, which in turn can be used to determine the dimension of axial deployment and outward deployment of the loop electrode 715 which in turn determines the cutting depth. In the variation shown in FIG. 17, the controller 140 is also coupled to a touch screen display 795 where the physician can select operating parameters of the systems, for example, the selected cutting depth. In this variation, the actuator button 796 in the handle 724 is adapted only for activating the loop electrode 715 while the touch screen display 795 is used for selecting other operational parameters.

In the cut-away view of FIG. 22, the dimensions of components of the working end 716′ are shown that provide for maximizing the cutting depth of the loop electrode 715. As can be understood, referring to FIG. 22, the objective is to position the rotational axis 726 of the electrode shaft 720 as close as possible to the outer surface of the introducer member 710 that defines introducer envelope 722 indicated at D3, which is less than 0.010″ or less than 0.050″. However, the outer sleeve 760 comprises return electrode 765, so that suitable insulative components must separate the electrode shaft 720 from the outer sleeve 760 to prevent electrical coupling within the wall of the introducer member 710. In a variation, the dialectic material in FIG. 22 has a dimension D4 of at least 0.003″ or at least 0.005″ extending through the introducer member 710 that comprises the wall thickness of the core sleeve 740 around the channel 732 and optionally the thickness of a heat shrink member 778 encasing the electrode shaft 720 (see FIG. 20). The resection devices described above can be used in various surgical fields such as urology, gynecology, arthroscopy, and other fields where resecting procedures are performed in a saline-immersed working space, such as in FIGS. 7A-7D and 13A-13D. For example, the RF devices and methods described above can be used in a BPH procedure to resect tissue to reduce the volume of a patient's prostate. In such a procedure, an articulating working end may not be needed, and a straight, rigid introducer may be used. Small-diameter RF devices, as described above, can be used in the field of arthroscopy, for example, to resect tissue in a patient's shoulder, hip, or knee again without an articulating working end.

In the variations described above, the negative pressure source 135 (FIGS. 1 and 8) communicates with a passageway in the introducer wherein the fluid inflow port is either at the distal end of the introducer or in a slot in the introducer from which the RF loop electrode is extended. It should be appreciated that the controller 140, or a fluid management system, typically will be used to control fluid pressure in the working space. Thus, the openings or ports in the working end of the introducer can be dimensioned to accommodate the required fluid flows, and additional ports may be disposed on any side of the working end to provide for adequate fluid outflows, wherein fluid inflows typically flow an inflow channel in the endoscope FIG. 7A).

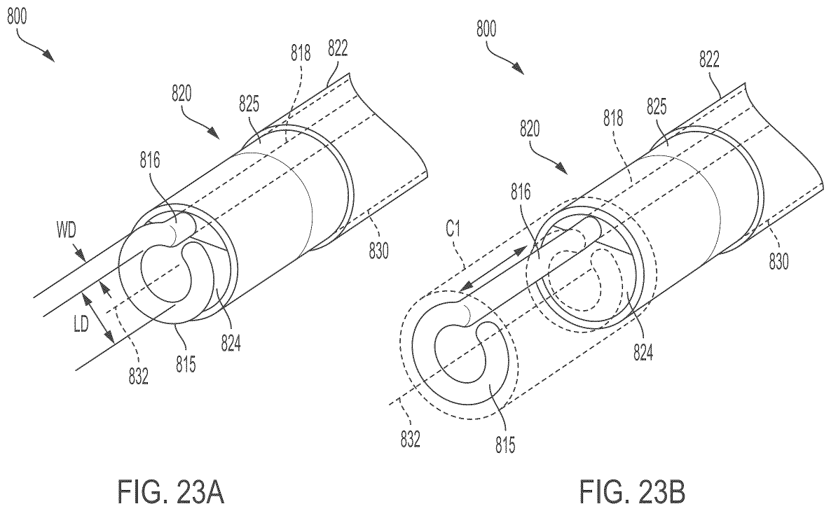

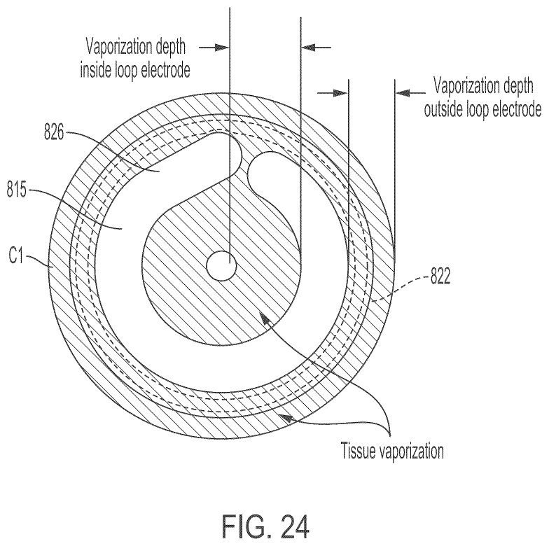

FIGS. 23A, 23B, and 24 illustrate another variation of a working end 800 of an electrosurgical resecting device that is similar to devices 650 and 700 of FIGS. 16 and 17, respectively. In the variation of FIGS. 23A and 23B, the RF loop electrode 815 is configured for axial movement to ablate or core a cylindrical path in targeted tissue wherein substantially all tissue in the path is vaporized, thus leaving very small tissue remnants or no tissue remnants at all. The vaporization of targeted tissue is possible (ii) when the diameter of the loop electrode 815 is small in relation to the electrode wire diameter, (ii) when the power delivered to the RF loop electrode is suitably high, and (iii) when the rate of axial movement of the electrode 815 in targeted tissue is at a suitable selected rate.

As can be seen in FIG. 23A, the RF loop electrode 815 again comprises a wire loop at the distal end of an axially-extending electrode shaft 816 that extends through bore 818 in an introducer assembly 820 that comprises an outer introducer sleeve 822 and a movable electrode-carrying inner sleeve 825 that carries the loop electrode 815. In a variation, FIG. 23A illustrates the loop electrode 815 having a wire diameter WD ranging from 0.020″ (0.50 mm) to 0.10″ (2.54 mm) and a mean inner loop diameter LD ranging from 2.5 mm to 5.0 mm. The loop electrode 815 is typically round but also may be oval or have angled regions, as in the variation of FIG. 24 below, where an angled portion 826 transitions to the axial-extending electrode shaft. For this reason, the inner loop diameter LD is defined as the mean dimension across the loop. Similarly, the wire forming the loop electrode 815 can be round, oval, rectangular, polygonal, or star-shaped in cross-section, and the wire diameter WD is defined as the mean dimension of a cross-section of the wire. In a variation, the ratio of the mean inner loop diameter LD to the wire diameter WD is 20:1 or less. In other variations, the ratio of the inner loop diameter LD to the wire diameter WD is 10:1 or less. In other variations, the ratio of the inner loop diameter LD to the wire diameter WD is 8:1 or less. In other variations, the ratio of the inner loop diameter LD to the wire diameter WD is 6:1 or less. The outer diameter of the loop electrode 815 is no larger than the outer diameter of the outer introducer sleeve 822 to allow introducer assembly to be introduced through a working channel of an endoscope as described in previous embodiments. Optionally, the outer diameter of the loop electrode 815 can be dimensioned for retraction into the lumen 828 of the outer sleeve 822 or retraction into the passageway 830 in the inner sleeve 825.

FIGS. 23B and 24 schematically illustrate the cylindrical hole or core C1 that the loop electrode 815 will bore into tissue about central axis 832. The end view of FIG. 24 further illustrates the dimensions of tissue ablation or vaporization around the loop electrode 815. It can be seen schematically that the tissue vaporization dimensions will result in a cylindrical hole for core C1 in targeted tissue as the loop electrode 815 is moved axially into tissue, as shown in FIG. 23B.

In a method of use, the loop electrode of FIGS. 23A, 23B, and 24 can be moved or advanced into targeted tissue in one of several modes of operation. First, as shown in FIG. 23B, the electrode shaft 816 and loop electrode 815 can be extended outwardly from bore 818 in the inner sleeve 825 either manually or by a motor drive to bore into targeted tissue. Second, the inner sleeve 825 carrying the loop electrode 815 can be extended outwardly from passageway 830 in the outer sleeve 822 either manually or by a motor drive to bore into targeted tissue. Third, the entire introducer assembly 820 can be moved and extended outwardly from a working channel of an endoscope, which would typically be a manual mode of operation. In any mode of operation, the negative pressure source 135 (FIG. 17) in communication with lumen 824 in the inner sleeve 825 will assist in removing any vaporized tissue or tissue remnants from the treatment site. Bore 818 can extend axially through inner sleeve 825.

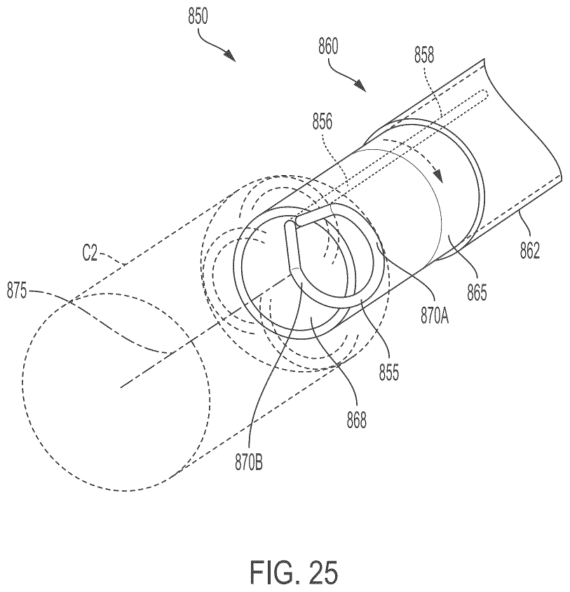

Now, turning to FIG. 25, another variation of a working end 850 of a resecting device is shown, which is similar to the variation of FIGS. 23A and 23B, except that an additional motor drive is provided to move the loop electrode 855 to vaporize a larger core or cylindrical hole in targeted tissue. In the variation of FIG. 25, the RF loop electrode 855 again comprises a wire loop at the distal end of an axially-extending electrode shaft 856 that extends through bore 858 in an introducer assembly 860 that comprises an outer introducer sleeve 862 and a movable inner sleeve 865 that carries the loop electrode 855. In this variation, the loop electrode 855 is optionally configured with the wire dimensions and loop dimensions of FIGS. 23A-23B to vaporize substantially all tissue. Alternatively, the loop electrode dimensions can be configured for cutting a path in tissue that leaves tissue chips or remnants that are extracted through the passageway 868 in the inner sleeve 865 by a negative pressure source (not shown). As can be seen in FIG. 25, the loop electrode 855 and its shaft 856 can be rotated outwardly from the introducer envelope, as defined above and locked in a selected position. The rotation of electrode shaft 856 is typically a manual operation, but motor-driven rotation is possible. In this variation, the outward rotation of the loop electrode 855 is limited to a degree of rotation, for example, up to approximately 90°, wherein a leading edge 870A of the loop electrode 855 is radially outward from the introducer envelope and an opposing trailing edge 870B the loop electrode 855 is inward from, or in alignment with, the central axis 875 of the inner sleeve 865. FIG. 25 then shows that at least one motor drive (cf. FIGS. 16 and 17) is used to (i) rotate the inner sleeve 865 within the outer sleeve 862 and (ii) contemporaneously move the entire assembly of inner sleeve 865 and RF loop electrode 855 axially in the distal direction to cut or vaporize a hole in tissue indicated dimensionally as core C2 in FIG. 25. Thus, the motor-driven variation of FIG. 25 will cut or vaporize tissue in a diameter substantially larger than the introducer envelope, whereas the variation of FIGS. 23A and 23B only vaporized a core C1 (see FIG. 23B), approximating the diameter of the introducer envelope.

In FIG. 25, it can be understood that rotation and the axial movement of loop electrode 855 will cut a spiral path in the tissue, leaving thin tissue strips that can be extracted through the passageway in the inner sleeve 865 by a negative pressure source. The rate of axial movement of the inner sleeve 865 will determine the dimension or thickness of the tissue strips which can be substantially small to be extracted through the interior aspiration passageway 868 in the inner sleeve 865. In another variation, a controller can operate the movement of the inner sleeve 865 to alternatively rotate the inner sleeve with and without axial movement to further facilitate cutting small tissue chips. In another variation, a motor drive, or manual actuator, can be used to rotate the electrode shaft 856 and loop electrode 855 inwardly and outwardly during rotation and axial movement to further facilitate cutting tissue chips of a small dimension for extraction through the aspiration passageway 868 in the inner sleeve 865. While at least one motor drive is described in the methods above, it should be appreciated that a single motor drive can be used to rotate the inner sleeve 865, and manual actuation can be used for advancing and retracting the inner sleeve 865 while cutting tissue.

In viewing FIG. 25, it can be understood that if the loop electrode 855 and shaft 856 were rotated outwardly at an angle greater than approximately 90° so that the trailing edge 870B of the loop electrode 855 was outward of the central axis 875, then contemporaneous rotation and axial movement of the inner sleeve 865 carrying a loop electrode 855 would not ablate, vaporize or resect tissue around the central axis 875 but instead create a donut-shaped hole in tissue.

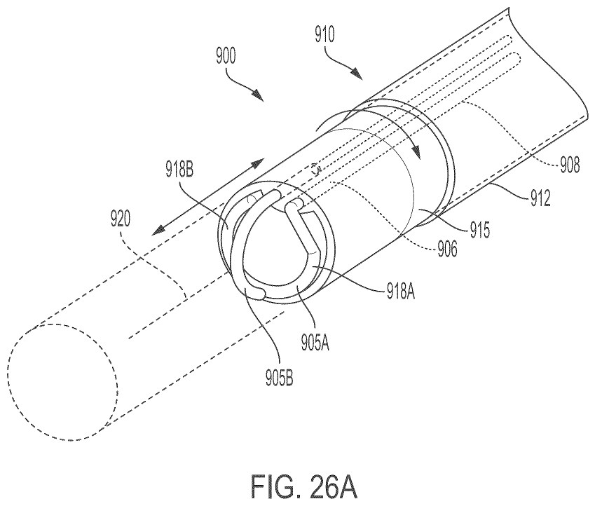

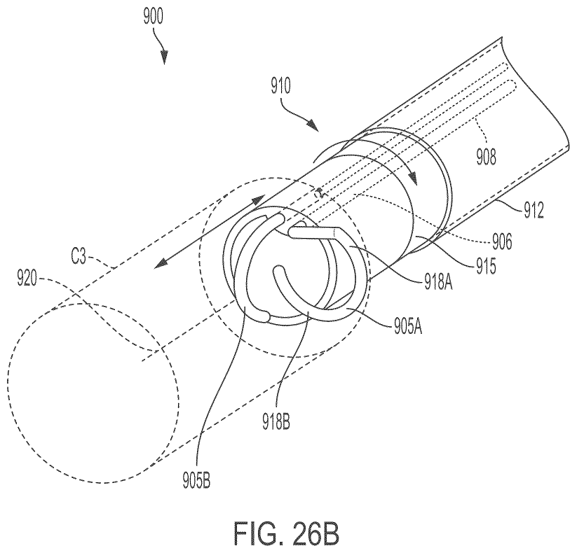

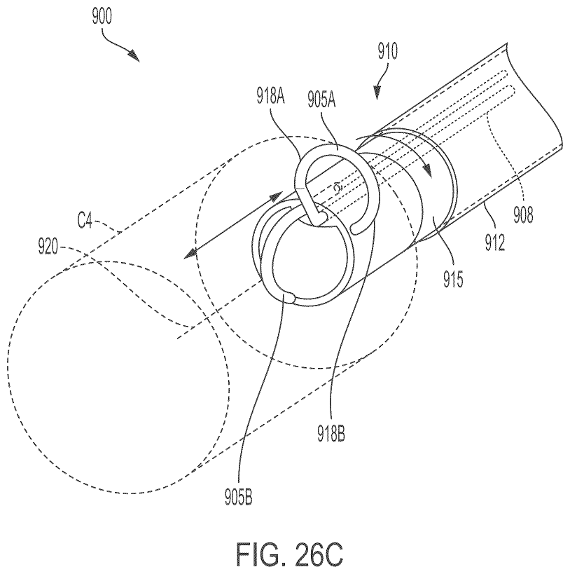

Referring now to FIGS. 26A-26C, another variation of a working end 900 of a resecting device is shown that is similar to that of FIG. 25, except that the active electrode arrangement comprises a moveable loop electrode 905A as described above and a secondary fixed electrode 905B that when operating together allow for ablating larger diameter cores in tissue. This variation again comprises an RF wire loop electrode 905A at the distal end of an axially-extending electrode shaft 906 that extends through bore 908 in an introducer assembly 910 that comprises an outer introducer sleeve 912 and a movable electrode-carrying inner sleeve 915 that carries the loop electrode 905A. As can be seen in FIG. 26A, the loop electrode 905A is similar to the variation of FIG. 25, except that the loop can be rotated further outward relative to the inner sleeve, for example, 180°, so that both the leading edge 918A and the trailing edge 918B of the electrode 905A are outward of the introducer envelope. No portion of the loop electrode is close to central axis 920 of the inner sleeve assembly. However, the secondary electrode 905B is fixed to the inner sleeve 915 and extends across the central axis 920. Therefore, rotation of the inner sleeve assembly will always cut tissue across the central axis 920 with the secondary fixed electrode 905B. At the same time, the loop electrode 905A can be rotated outside the introducer envelope, as shown in FIGS. 26B and 26C to cut a substantially larger path in tissue, as indicated by cores C3 and C4 in FIGS. 26B and 26C, respectively.

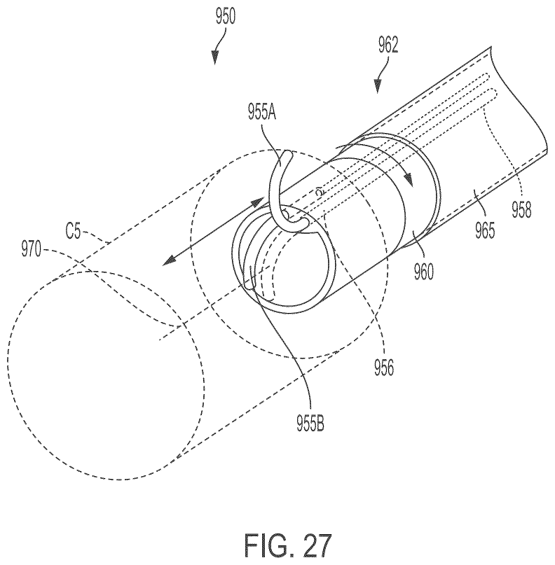

FIG. 27 illustrates another variation of a working end 950 of a resecting device that is similar to the variation of FIGS. 26A-26C, except that the rotatable electrode 955A has an arc-shape instead of a loop shape. The rotatable electrode 955A has an axially-extending electrode shaft 956 that extends through bore 958 in an inner sleeve 960 of an introducer assembly 962. The outer introducer sleeve 965 is similar to previous variations. The secondary fixed electrode 955B also has an arc shape that extends across the central axis 970, but it should be appreciated that the fixed electrode can have any shape, such as an elbow or “L” shape, that extends to or across the central axis 970. As can be seen in FIG. 27, a core C5 can be cut with the electrode 955A fully rotated outwardly.

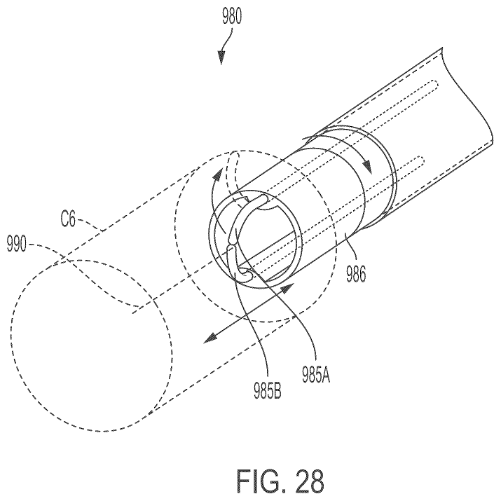

FIG. 28 illustrates another variation of a working end 980 that is similar to the variation of FIG. 27 except that the rotatable electrode 985A and fixed electrode 985B extend from opposing sides of the inner sleeve 986, with each electrode having an arc shape that extends to the central axis 990. This variation of working end 980 will resect tissue in the outline of core C6 with the rotatable electrode 985A rotated 180° outward, followed by rotation and axial movement of the inner sleeve 986 carrying electrode 985A.

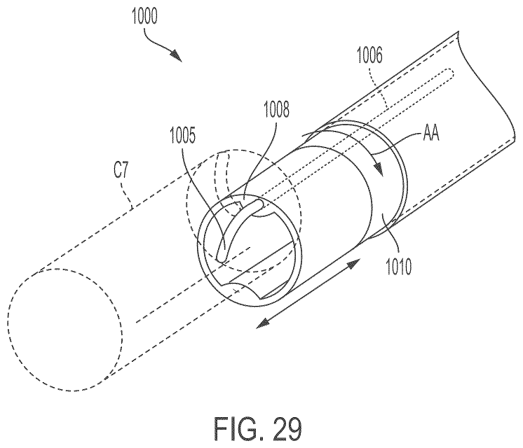

FIG. 29 illustrates another variation of a working end 1000 with an arc-shaped electrode 1005 that is motor-driven to rotate in 360° within bore 1006 in a wall 1008 of the inner sleeve 1010. FIG. 29 illustrates that core C7 would be resected by electrode 1005 when rotated in bore 1006 together with axial movement of the inner sleeve 1010. It can be understood that if the inner sleeve 1010 was also rotated around its central axis 1015 as indicated by arrow AA, then the resected core would be far larger and similar to the core C6 of FIG. 28. In a method of use, the rate of rotation of the electrode 1005, the rate of rotation of the inner sleeve 1010, and the rate of axial movement of the inner sleeve 1010 can vary or be intermittent to achieve suitable rates of tissue resection.

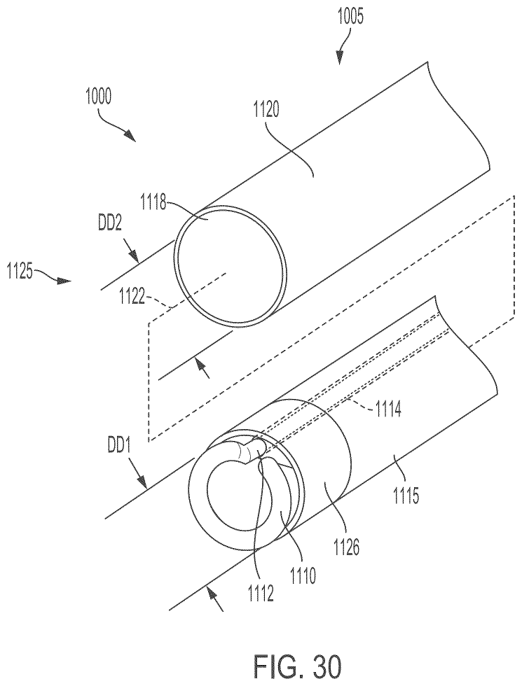

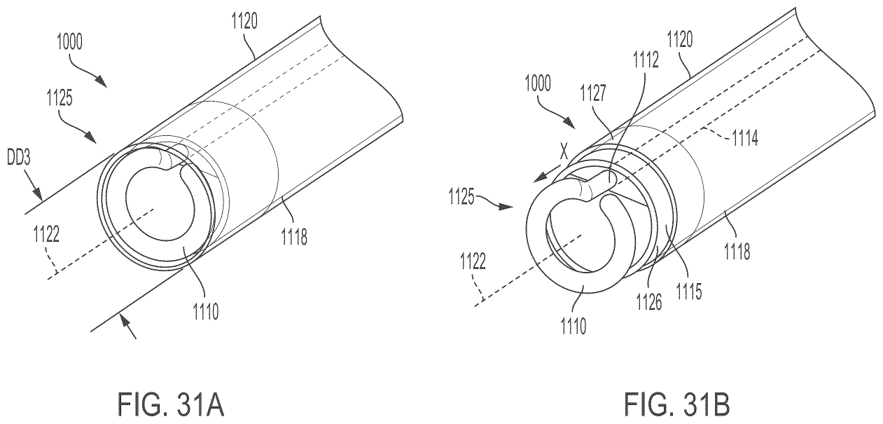

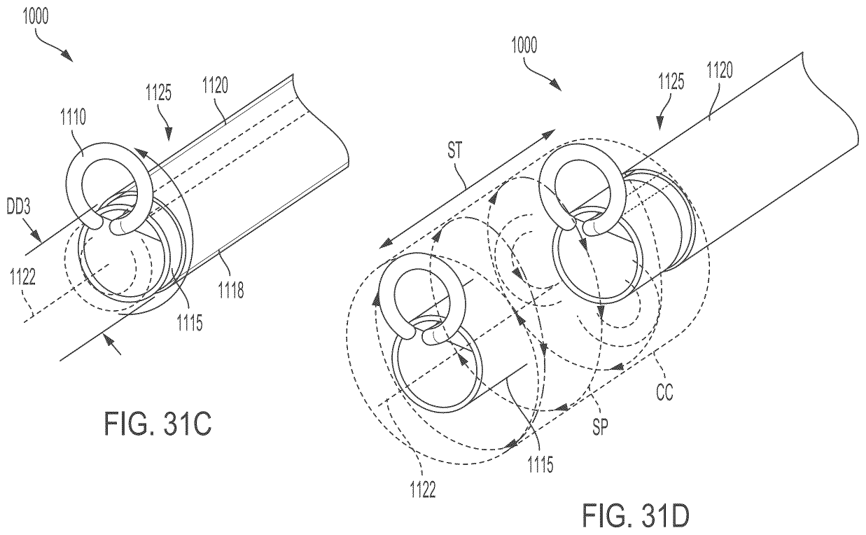

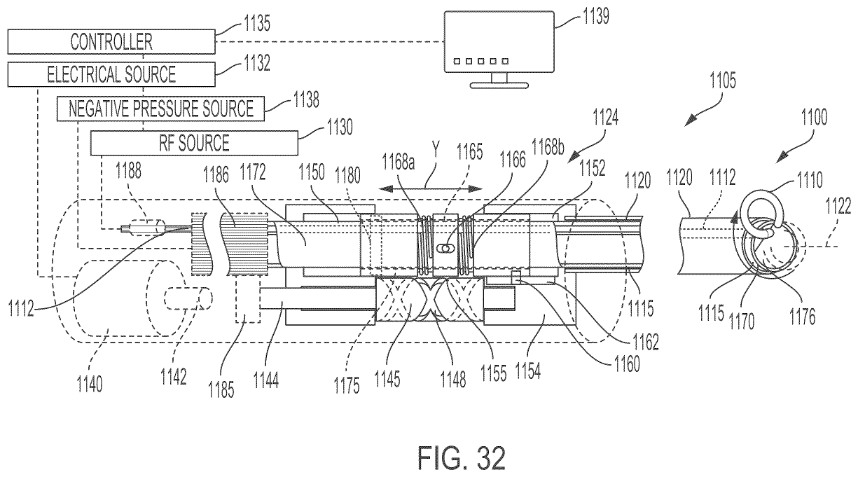

FIGS. 30 and 31A-31D illustrate another variation of a working end 1100 of a resecting device 1105, which is similar to previous variations. In the variation of FIG. 30, the RF loop electrode 1110 again comprises a wire loop at the distal end of an axially-extending electrode shaft 1112 that extends through an off-center bore 1114 in a cutting sleeve 1115 that is moveable in a bore 1118 in an outer introducer member or sleeve 1120. The introducer assembly 1125 comprises the introducer sleeve 1120 and the cutting sleeve 1115 that extend about central axis 1122 from a handle 1124 (see FIG. 32). FIG. 30 shows the introducer sleeve 1120 and cutting sleeve 1115 de-mated from one another. In FIG. 30, it can be seen that the outer diameter DD1 of the loop electrode 1110 approximates outer diameter of the cutting sleeve 1115 and the diameter DD2 of bore 1118 in the introducer sleeve 1120 so that the loop electrode can be fully retracted into the bore 1118 as shown in FIG. 31A. Thus, the outer diameter of DD1 of the loop electrode 1110 is as large as possible to maximize cutting depth when the loop electrode 1110 is rotationally deployed. The loop electrode 1110 is an active electrode, and a distal exterior surface of cutting sleeve 1115 is configured with a return electrode 1126 as described previously. In this variation, the outer diameter of the introducer sleeve 1120 is from 2.5 mm to 10 mm, and often 5 mm is diameter or less. The RF loop electrode 1110 comprises a wire having a diameter ranging from 0.015″ to 0.025.

FIG. 31A shows the introducer sleeve assembly 1125 with inner cutting sleeve 1115 retracted within the bore 1118 of introducer sleeve 1120 in a first position or default position for introducing the working end 1100 through a working channel of an endoscope as described previously. It can be seen that the introducer sleeve 1120 has an outer diameter extending distally that defines an introducer envelope DD3 as described above. FIG. 31B illustrates another position of the cutting sleeve 1115 and loop electrode 1110 wherein the loop electrode is extended distally from the bore 1118 of introducer sleeve 1120 a short distance X so that the loop electrode 1110 is distal from the distal end 1127 of the introducer sleeve 1120. From the position shown in FIG. 31B, it can be understood that the loop electrode 1110 can be rotated by an actuator 128 in the handle 1124 (FIG. 32) to position the loop electrode 1110 outwardly from the introducer envelope DD3. In a variation, the loop electrode 1110 can be rotated outward and locked in a plurality of selected positions, for example, 60°, 90°, and 180°. FIG. 31C shows the loop electrode 1110 after being rotated outwardly 180° from the cutting sleeve 1115 to provide a maximum cutting depth. With the loop electrode 1110 in the position shown in FIG. 31C, the physician can then manually move the working with the RF source 1130 providing current in a coagulation mode for coagulating tissue or in a cutting mode for cutting tissue.

FIG. 31D shows the working end 1100 in an automated cutting mode wherein the inner cutting sleeve 1115 and loop electrode 1110 are motor-driven to extend and retract from the introducer sleeve 1120 in a spiral path SP. In other words, at least one motor drive is actuated to contemporaneously rotate and reciprocate the cutting sleeve 1115 which results in cutting the spiral path SP in tissue. In the variation of FIG. 31D, it can be seen that the spiral path SP includes 3 revolutions of 360° of the energized RF loop electrode 1110, which then cuts a cylindrical envelope indicated at CC. The motor-driven system can be adapted to rotate the cutting sleeve 1115 from 1 revolution to 10 revolutions or more which then defines a stroke ST having an axial length of from 5 mm to 20 mm (FIG. 31C). In some variations, the spiral path SP can comprise between two to eight revolutions of 360° of the energized RF loop electrode 1110.

In a variation, the motor-driven system can rotate the cutting sleeve 1115 and RF loop electrode 1110 in a first rotational direction when being extended and in an opposing rotational direction when being retracted. In another variation, the cutting sleeve 1115 and loop electrode 1110 can be rotated in a single rotational direction when being extended and when being retracted.

In another variation, a first motor drive can be adapted to reciprocate the cutting sleeve 1115 and loop electrode 1110 at a selected speed, and a second motor drive can be adapted to rotate the cutting sleeve 1115 at an independent selected speed. In such a variation, the pitch of the spiral path SP, along with the rate of extension and retraction, can be selected to optimize the spiral movement of the RF loop electrode 1110 for cutting tissue.

Now, turning to FIG. 32, a schematic view of a handle 1124 of the resecting device 1105 of FIGS. 30-31D is shown together with a diagram of the RF source 1130, electrical source 1132, controller 1135, and negative pressure source 1138 coupled to the resection device 1105. A touch screen 139 is also shown, which can be used to select different modes of operation. In this variation, a single electrical motor drive 1140 is coupled to electrical source 1132 and is adapted to rotate the motor shaft 1142, which is coupled to drive shaft 1144 of a rotating cylindrical cam member 1145. A continuous advancing and reversing spiral groove 1148 is provided in the cam surface as is known in the art for reciprocating an adjacent member. In this variation, a cooperating reciprocating sleeve 1150 is adapted to move axially in channel 1152 in handle housing 1154, wherein the sleeve 1150 carries an elliptically-shaped slider 1155 that slides within the spiral groove 1148 of the cam member 1145 so that the elliptically-shaped slider 1155 caused reciprocating axial motion of the slider and sleeve 1150 in response to rotation of the cylindrical cam member 1145.

The reciprocating sleeve 1150 also carries a key 1160 that slides in slot 1162 in housing 1154 so that the sleeve moves back and forth in channel 1152 without rotating (see arrow Y in FIG. 32).

In the variation of FIG. 32, the elliptically-shaped slider 1155 is carried in a thin-wall collar 1165 that is keyed with pin 1166 to axially slide around reciprocating sleeve 1150. The assembly of the collar 1165 and pin 1166 and springs 1168a and 1168b on opposing sides of the collar 1165 allow slight axial movement of the collar as a shock-absorbing mechanism. This shock-absorbing mechanism is optional and can be useful when cutting tissue that may be too hard to penetrate.

In the variation of FIG. 32, it can be seen that a distal portion 1170 of the cutting sleeve 1115 rotates and reciprocates in the introducer sleeve 1120 that is fixed in the handle housing 1154. The proximal portion 1172 of the cutting sleeve 1115 extends through the handle 1124 and, more particularly, through the bore 1175 of the reciprocating sleeve 1150. The negative pressure source 1138 communicates with the interior passageway 1176 in the cutting sleeve 1115, as described previously. As can be seen in FIG. 32, the cutting sleeve 1115 has an annular projecting ring 1180 that rotates in an annular groove in the bore 1175 of the reciprocating sleeve 1150 so that reciprocation of the reciprocating sleeve 1150 also reciprocates the cutting sleeve 1115.

Rotation of the cutting sleeve 1115 is provided by the motor drive 1140 that rotates a gear first gear 1185 on the cam drive shaft 1144 that in turn engages a second gear 1186 fixed on a proximal portion 1172 of the cutting sleeve 1115. Thus, the spiral cam member 1145 and the gears 1185 1186 impart both reciprocating motion and rotational motion to the cutting sleeve 1115. In this variation, the rotational speed of the cutting sleeve 1115 is in a fixed relationship with the rate of reciprocation due to the fixed gearing. In another variation, a first motor drive can be used in the reciprocation mechanism and a second motor drive can rotate the cutting sleeve, which would allow for a variable relationship between reciprocation and rotation, thus allowing for varied pitches of the spiral path of the loop electrode 1110.

In FIG. 32, the electrode shaft 1112 is shown extending through the handle 1124 to a grip 1188 that can be manually operated to move the loop electrode 1110 from its default position of FIG. 31A to the extended position FIG. 31B and then to any rotated position as shown in FIG. 31C. The grip 1188 is also adapted for locking the loop electrode 1110 in a selected deployed position. FIG. 32 also schematically shows the RF source 1130 coupled to the electrode shaft 1112 in the handle 1124. In this variation, there also is a contact switch (not shown) in the handle that prevents RF source 1130 from delivering current to the electrode 1110 in the default position of FIG. 31A.

In another variation, the controller 1135 is configured to fully control movement of the loop electrode 1110 from the default position of FIG. 31A through the spiral cutting mode, as shown in FIG. 31D. In such a variation, a contact switch in the handle or an encoder on the motor shaft prevents the RF source 1130 from delivering current to the loop electrode 1110 in the default position. After the device 1105 is actuated by a switch in the handle 1124 or a foot switch, the controller 1135 then operates the motor drive 1140 to move the loop electrode 1110 axially and then to a selected outward position as in FIG. 31D, and then move the electrode in a spiral path back and forth in a selected stroke to electrosurgically resect tissue. In this variation, the controller 1135 always returns the loop electrode 1110 to default or beginning position, wherein the RF loop electrode 1110 is within the introducer envelope DD3 to allow its withdrawal through the working channel of an endoscope. In this variation, the touch screen 1139 can be configured to allow for touch screen selection of all the various operating parameters of the resection device, which can include (i) the rate of axial movement of the loop electrode 1110, (ii) the rotational speed of the loop electrode 1110, (iii) the stroke of the loop electrode 1110, and (iv) the outer diameter of the cylindrical envelope CC (FIG. 31D) cut by the loop electrode 1110 as shown in FIG. 31D.

Although particular variations of the present invention have been described above in detail, it will be understood that this description is merely for purposes of illustration, and the above description of the invention is not exhaustive. The variations above are shown with a motor drive for moving a cutting sleeve helically and rotationally, but manually operated mechanisms are also possible for either or both such helical and rotational movements. Specific features of the invention are shown in some drawings and not in others, and this is for convenience only, and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Various changes may be made to the invention described, and equivalents (whether recited herein or not included for the sake of some brevity) may be substituted without departing from the true spirit and scope of the invention. Also, any optional feature of the inventive variations may be set forth and claimed independently, or in combination with any one or more of the features described herein. Accordingly, the invention contemplates combinations of various aspects of the embodiments or combinations of the embodiments themselves, where possible. Reference to a singular item includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

What is claimed is:

1. An electrosurgical device, comprising:
a handle coupled to an elongated introducer member extending about a central axis to a working end, wherein the elongated introducer member has an outer surface that defines an introducer envelope;
an RF loop electrode carried at a distal end of a cutting sleeve extending through a bore in the elongated introducer member to the handle;
an actuator in the handle adapted for moving the RF loop electrode between a first position within the introducer envelope and at least one second position radially outward from the introducer envelope; and
a motor-driven mechanism in the handle configured for extension and retraction of the cutting sleeve in a spiral path from a distal end of the bore of the elongated introducer member.

2. The electrosurgical device of claim 1, wherein the spiral path includes at least one 360° rotation of the cutting sleeve and RF loop electrode.

3. The electrosurgical device of claim 1, wherein the spiral path comprises from two to eight 360° rotations of the cutting sleeve and RF loop electrode.

4. The electrosurgical device of claim 1, wherein the spiral path defines a stroke having an axial length that ranges from 5 mm to 20 mm.

5. The electrosurgical device of claim 1, wherein the motor-driven mechanism provides a fixed pitch of the spiral path.

6. The electrosurgical device of claim 1, wherein the motor-driven mechanism allows for selection of pitch of the spiral path.

7. The electrosurgical device of claim 1, wherein the motor-driven mechanism comprises a first motor drive for rotating the cutting sleeve and a second motor drive for reciprocating the cutting sleeve.

8. The electrosurgical device of claim 1, wherein the motor-driven mechanism rotates the cutting sleeve in a first rotational direction in the extension of the cutting sleeve and an opposing rotational direction in the retraction of the cutting sleeve.

9. The electrosurgical device of claim 1, wherein the motor-driven mechanism rotates the cutting sleeve in the same rotational direction in the extension of the cutting sleeve and in the retraction of the cutting sleeve.

10. The electrosurgical device of claim 1, wherein the actuator is adapted to move the RF loop electrode to a plurality of selected positions radially outward from the introducer envelope.

11. The electrosurgical device of claim 1, wherein the actuator in the handle is manually operated.

12. The electrosurgical device of claim 1, wherein the RF loop electrode in the first position is retracted into the bore of the elongated introducer member.

13. The electrosurgical device of claim 1, wherein an outer diameter of the elongated introducer member is 5 mm or less.

14. The electrosurgical device of claim 1, wherein an outer diameter of the elongated introducer member is from 2.5 mm to 10 mm.

15. The electrosurgical device of claim 1, wherein the RF loop electrode comprises a wire having a diameter 0.015″ to 0.025″.

16. An electrosurgical device, comprising:
an introducer member extending about a central axis to a working end, wherein the introducer member has an outer surface that defines an introducer envelope;
an RF loop electrode with a carried at the working end;
wherein the RF loop electrode comprises a wire formed into a loop with a mean loop diameter across an interior of a loop of the RF loop electrode; and
wherein a ratio of the mean loop diameter to a diameter of the wire is 20:1 or less.

17. The electrosurgical device of claim 16, wherein the ratio of the mean loop diameter to the diameter of the wire is 10:1 or less.

18. An electrosurgical device, comprising:
a handle coupled to an elongated introducer assembly extending about a central axis to a working end, wherein the elongated introducer assembly has an outer surface that defines an introducer envelope;
a bi-polar RF electrode arrangement carried at the working end including an RF electrode at a distal end of an electrode shaft extending through an axial bore in a wall of the elongated introducer assembly; and
an actuator in the handle configured to rotate the electrode shaft to deploy the RF electrode from a non-deployed position within the introducer envelope to a deployed position wherein a portion of the RF electrode is radially outward from the introducer envelope.

19. The electrosurgical device of claim 18, wherein a portion of the RF electrode in the deployed position is inward of the central axis.

20. The electrosurgical device of claim 18, wherein the RF electrode has a shape selected from group of loop shapes, curved shapes, angled shapes and linear shapes.

21. The electrosurgical device of claim 18, further comprising a motor drive adapted to rotate the elongated introducer assembly.

22. The electrosurgical device of claim 18, further comprising a motor drive adapted to move the elongated introducer assembly axially.

23. The electrosurgical device of claim 18, further comprising a motor drive adapted to rotate the electrode shaft.

24. The electrosurgical device of claim 18, further comprising a negative pressure source in communication with a passageway in the elongated introducer assembly.

25. An electrosurgical tissue resection method, comprising:
providing a device comprising a handle coupled to an elongated introducer having an outer surface that defines an introducer envelope, an RF loop electrode carried at a working end of the elongated introducer, and an actuator in the handle adapted for rotating a shaft of the RF loop electrode;
introducing the working end into a working space;
actuating the actuator to deploy the RF loop electrode from a non-deployed position within the introducer envelope to a deployed radially outward from the introducer envelope; and
energizing the RF loop electrode and moving the RF loop electrode to thereby resect tissue in the working space.

26. The electrosurgical tissue resection method of claim 25, wherein the RF loop electrode is moved axially to resect tissue.

27. The electrosurgical tissue resection method of claim 25, wherein the RF loop electrode is moved axially by at least one of manually and by a motor drive.

28. The electrosurgical tissue resection method of claim 25, wherein the RF loop electrode is moved rotationally to resect tissue.

29. The electrosurgical tissue resection method of claim 25, wherein the RF loop electrode is moved rotationally by at least one of manually and by a motor drive.

30. The electrosurgical tissue resection method of claim 25, further comprising extracting resected tissue through a lumen in the elongated introducer responsive to negative pressure in the lumen provided by a negative pressure source coupled thereto.
